(19) United States
(12) Patent Application Publication
(10) Pub. No.: US 2023/0233168 A1
(43) Pub. Date: Jul. 27, 2023

(54) PULSE WAVE VELOCITY DETERMINATION USING CO-REGISTRATION BETWEEN INTRAVASCULAR DATA AND EXTRALUMINAL IMAGE, AND ASSOCIATED SYSTEMS, DEVICES, AND METHODS

(71) Applicant: KONINKLIJKE PHILIPS N.V., Eindhoven (NL)

(72) Inventors: Sara Rose CHEN, SAN DIEGO, CA (US); Becky INDERBITZEN, SAN DIEGO, CA (US); Arjen VAN DER HORST, TILBURG (NL)

(21) Appl. No.: 18/098,797

(22) Filed: Jan. 19, 2023

Related U.S. Application Data
(60) Provisional application No. 63/302,388, filed on Jan. 24, 2022.

(51) Int. Cl. A61B 6/00; A61B 5/02

(52) U.S. Cl. CPC A61B 6/50; A61B 5/02007

(57) ABSTRACT

A system includes a processor circuit configured to receive a set of intravascular data from an intravascular sensor at a first location within a blood vessel. The processor circuit simultaneously receives a set of cardiovascular data from a heart monitor. After the intravascular sensor is moved from the first location to a second location, the processor circuit receives an additional set of intravascular data from the intravascular sensor and an additional set of cardiovascular data from the heart monitor. The processor circuit then determines a distance between the first location and the second location and determines a pulse wave velocity associated with the blood flow within the blood vessel based on the sets of intravascular data, the sets of cardiovascular data, and the distance. The processor circuit then outputs the pulse wave velocity to a display.

### CROSS-REFERENCE TO RELATED APPLICATIONS

This application claims priority to and the benefit of U.S. Provisional Application No. 63/302,388, filed Jan. 24, 2022, which is incorporated by reference herein in its entirety.

### TECHNICAL FIELD

The present disclosure relates generally to pulse wave velocity measurement. In particular, pulse wave velocity within a vessel is determined with an intravascular device with a single hemodynamic sensor while the device is tracked using coregistration.

### BACKGROUND

Physicians use many different medical diagnostic systems and tools to monitor a patient's health and diagnose medical conditions. In the field of cardiovascular health in patients, various systems and devices are used to monitor a patient's condition and perform treatment procedures. A pulse-wave velocity (PWV) is a measurement of the rate at which pressure waves move through the patient vasculature. PWV measurements assist a physician in determining arterial stiffness and can serve as a predictor of cardiovascular risk. PWV measurements also help physicians assess the cardiovascular health of patients with renal disease, diabetes and hypertension. PWV measurements are also affected by changes in a patient's sympathetic nervous system response to various stimuli. As a result, PWV measurements may be used to quantify a patient's sympathetic response and stratify patients for renal denervation procedures. Additionally, PWV measurements may quantify the effect of a renal nerve ablation procedure or a renal nerve stimulation procedure via alterations in the measured PWV.

PWV measurements may be obtained invasively or non-invasively. However, invasive PWV measurement procedures typically produce more accurate PWV measurements and are therefore more reliable. A typical invasive pulse wave velocity measurement procedure requires an intravascular device with at least two hemodynamic sensors spaced apart by some known distance. These two sensors may obtain blood pressure data, blood flow data, or other data. As a blood pulse wave passes by each of the sensors, the time at which the wave passed each sensor may be recorded. The difference in time and the distance between the sensors is used to determine the velocity of the pulse wave. While this method of determining PWV is accurate and reliable, it requires a separate, more specialized intravascular device than most common intravascular devices. As a result, for a catheter lab to obtain invasive pulse wave velocity measurements, an additional intravascular device must be purchased and a separate intravascular procedure must be performed, meaning one device must be removed from the patient anatomy and the pulse wave velocity device must then be positioned.

### SUMMARY

Embodiments of the present disclosure are systems, devices, and methods for calculating a pulse wave velocity measurement using coregistration between intravascular data and an extraluminal image. Aspects of the present invention advantageously provide a physician with a way to accurately determine the pulse wave velocity of any location within a patient vasculature using various common intravascular devices. In particular, the pulse wave velocity may be determined using an intravascular device with one data sensor. The one data sensor may be a pressure sensor, a flow sensor, an intravascular ultrasound imaging sensor, or any other type of sensor.

In one aspect of the disclosure, the intravascular device is positioned at one location and acquires intravascular data while a heart monitor acquires cardiovascular data relating to the cardiac cycle of the patient and an extraluminal imaging system acquires extraluminal images showing the location of the intravascular device. The system identifies the location of the intravascular device. The system then selects a feature of the cardiovascular data, such as a minimum or maximum value, and determines the time at which the feature was obtained by the heart monitor. The system selects a feature of the intravascular data, such as a maximum value, and determines the time at which the feature was obtained by the intravascular device. The system then determines a difference in the time value of the feature of the cardiovascular data and the time value of the feature of the pressure data.

The intravascular device may then be positioned at a different location and the process may be repeated. In this way, the system may determine two locations of the intravascular data and corresponding time difference values for each respective location. The system may then determine a difference in the time difference values and a distance between the two locations. The system then may determine the pulse wave velocity between the two locations based on the distance between the two locations and the difference in the time differences of the two locations.

In an exemplary aspect, a system is provided. The system includes a processor circuit configured for communication with a display, a heart monitor, and an intravascular catheter or guidewire, wherein the processor circuit is configured to: receive, from the intravascular catheter or guidewire, a first set of intravascular data obtained by a single intravascular sensor of the intravascular catheter or guidewire while the intravascular catheter or guidewire is positioned at a first location within the blood vessel; receive, from the heart monitor, a first set of cardiovascular data obtained while the single intravascular sensor obtains the first set of intravascular data; receive, from the intravascular catheter or guidewire, a second set of the intravascular data obtained by the single intravascular sensor while the intravascular catheter or guidewire is positioned at a second location within the blood vessel; receive, from the heart monitor, a second set of the cardiovascular data obtained while the single intravascular sensor obtains the second set of intravascular data; determine a distance between the first location and the second location; determine a velocity of a pulse wave associated with blood flow within the blood vessel, based on the first set of intravascular data, the second set of intravascular data, the first set of cardiovascular data, the second set of cardiovascular data, and the distance; and provide, to the display, an output based on the velocity of the pulse wave.

In one aspect, the first set of the cardiovascular data and the second set of the cardiovascular data include electrocardiogram (ECG) data. In one aspect, the single intravascular sensor comprises a pressure sensor, and the first set of the intravascular data and the second set of the intravascular data comprise intravascular pressure data. In one aspect, the single intravascular sensor comprises a flow sensor, and the first set of the intravascular data and the second set of the intravascular data comprise intravascular flow data. In one aspect, the single intravascular sensor comprises an imaging sensor, and the first set of the intravascular data and the second set of the intravascular data comprise intravascular imaging data. In one aspect, the processor circuit is configured for communication with an extraluminal imaging device, the processor circuit is configured to receive one or more extraluminal images obtained by the extraluminal imaging device, and the processor circuit is configured to determine the distance based on the one or more extraluminal images. In one aspect, the processor circuit is configured for communication with an extraluminal imaging device, and the processor circuit is configured to determine the distance based on co-registration of at least one of the first set of intravascular data or the second set of intravascular data to one or more extraluminal images obtained by the extraluminal imaging device. In one aspect, the first set of the cardiovascular data corresponds to a first cyclic waveform; the first set of the intravascular data corresponds to a second cyclic waveform; the second set of the cardiovascular data corresponds to a third cyclic waveform; and the second set of the intravascular data corresponds to a fourth cyclic waveform. In one aspect, the processor circuit is further configured to: identify a first time at which a first feature of the first cyclic waveform occurs; identify a second time at which a second feature of the second cyclic waveform occurs; identify a third time at which a third feature of the third cyclic waveform occurs; identify a fourth time at which a fourth feature of the fourth cyclic waveform occurs; determine a first difference between the first time and the second time; and determine a second difference between the third time and the fourth time, and wherein the processor circuit is configured to determine the velocity of the pulse wave based on the first difference, the second difference, and the distance. In one aspect, the processor circuit is configured to determine a third difference between the first difference and the second difference, and the processor circuit is configured to determine the velocity of the pulse wave based on the third difference and the distance. In one aspect, to determine the velocity of the pulse wave, the processor circuit is configured to divide the distance by the third difference. In one aspect, the first feature and the third feature comprise a same feature of the cardiovascular data, and the second feature and the fourth feature comprise a same feature of the intravascular data. In one aspect, the blood vessel comprises a renal artery.

In an exemplary aspect, a method is provided. The method includes receiving, by a processor circuit in communication with an intravascular catheter or guidewire comprising only a single intravascular sensor, a first set of intravascular data obtained by the single intravascular sensor while the intravascular catheter or guidewire is positioned at a first location within the blood vessel; receiving, by the processor circuit, a first set of cardiovascular data while the single intravascular sensor obtains the first set of intravascular data, wherein the first set of cardiovascular data is obtained by a heart monitor in communication with the processor circuit; receiving, by a processor circuit, a second set of the intravascular data obtained by the single intravascular sensor while the intravascular catheter or guidewire is positioned at a second location within the blood vessel; receiving, by the processor circuit, a second set of the cardiovascular data obtained by the heart monitor while the single intravascular sensor obtains the second set of intravascular data; determining, by the processor circuit, a distance between the first location and the second location; determining, by the processor circuit, a velocity of a pulse wave associated with blood flow within the blood vessel, based on the first set of intravascular data, the second set of intravascular data, the first set of cardiovascular data, the second set of cardiovascular data, and the distance; and providing, by the processor circuit, an output based on the velocity of the pulse wave to a display in communication with the processor circuit.

In an exemplary aspect, a system is provided. The system includes an intravascular catheter or guidewire configured to be positioned within a blood vessel of a patient and comprising only a single intravascular sensor; and a processor circuit configured for communication with a heart monitor, an extraluminal imaging device, a display, and the intravascular catheter or guidewire, wherein the processor circuit is configured to: determine a first time difference between when a first feature occurs in a first set of electrocardiogram (ECG) data and when a second feature occurs in a first set of intravascular data, wherein the first set of the intravascular data is obtained by the single intravascular sensor at a first location within the blood vessel simultaneously as the first set of the ECG data is obtained by the heart monitor; determine a second time difference between when a third feature occurs in a second set of ECG data and when a fourth feature occurs in a second set of intravascular data, wherein the second set of the intravascular data is obtained by the single intravascular sensor at a second location within the blood vessel simultaneously as the second set of ECG data is obtained by the heart monitor; determine a distance between the first location and the second location based on one or more extraluminal images obtained by the extraluminal imaging device; determine a velocity of a pulse wave associated with blood flow within the blood vessel based on the distance, the first time difference, and the second time difference; and provide, to the display, an output based on the velocity of the pulse wave.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a diagrammatic schematic view of an exemplary intravascular system according to some embodiments of the present disclosure.

FIG. 2 is a diagrammatic view of an intravascular device positioned within the renal artery of a patient, according to aspects of the present disclosure.

FIG. 3 is a diagrammatic cross-sectional view of an example sensor assembly, according to aspects of the present disclosure.

FIG. 4 is a schematic diagram of a processor circuit, according to aspects of the present disclosure.

FIG. 5 is a diagrammatic view of a relationship between x-ray fluoroscopy images, intravascular data, and a path defined by the motion of an intravascular device, according to aspects of the present disclosure.

FIG. 6A is a diagrammatic view of an intravascular device within a lumen, according to aspects of the present disclosure.

FIG. 6B is a diagrammatic view of an intravascular device within a lumen, according to aspects of the present disclosure.

FIG. 7A is a diagrammatic view of an intravascular device according to aspects of the present disclosure.

FIG. 7B is a diagrammatic view of an intravascular device according to aspects of the present disclosure.

FIG. 8 is a diagrammatic view of an intravascular device within a region of a patient anatomy, according to aspects of the present disclosure.

FIG. 9 is a diagrammatic view of an intravascular device within a region of a patient anatomy, according to aspects of the present disclosure.

FIG. 10 is a diagrammatic view of an intravascular device within a region of a patient anatomy, according to aspects of the present disclosure.

FIG. 11 is a diagrammatic view of an ECG curve and a blood pressure curve associated with a time axis and acquired before an intravascular device is moved within a renal artery, according to aspects of the present disclosure.

FIG. 12 is a diagrammatic view of an ECG curve and a blood pressure curve associated with a time axis and acquired after an intravascular device is moved within a renal artery, according to aspects of the present disclosure.

FIG. 13 is a diagrammatic view of an intravascular device within a region of a patient anatomy, according to aspects of the present disclosure.

FIG. 14 is a diagrammatic view of an intravascular device within a region of a patient anatomy, according to aspects of the present disclosure.

FIG. 15 is a diagrammatic side view of an intraluminal sensing system that includes an intravascular device comprising conductive members and conductive ribbons, according to aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

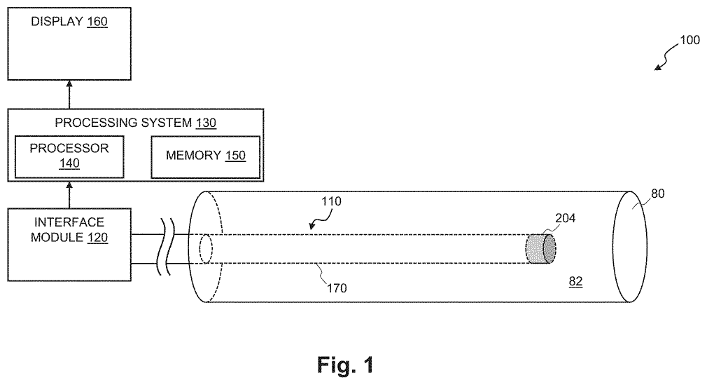

FIG. 1 is a diagrammatic schematic view of an exemplary intravascular system 100 according to some embodiments of the present disclosure. The intravascular system 100, which may be referred to as a stratification system, may be configured to perform pulse wave velocity (PWV) measurements in a vessel 80 (e.g., artery, vein, etc.), for patient stratification for treatment purposes. For example, the PWV determination in the renal arteries may be utilized to determine whether a patient is suitable for renal artery denervation. The intravascular system 100 may include an intravascular device 110 that may be positioned within the vessel 80, an interface module 120, a processing system 130 having at least one processor 140 and at least one memory 150, and a display 160.

In some embodiments, the system 100 may be configured to perform pulse wave velocity (PWV) determination in a vessel 80 within a body portion. The intravascular system 100 may be referred to as a stratification system in that the PWV may be used for patient stratification for treatment purposes. For example, the PWV determination in the renal arteries may be utilized to determine whether a patient is suitable for renal artery denervation. Based on the PWV determination, the intravascular system 100 may be used to classify one or more patients into groups respectively associated with varying degrees of predicted therapeutic benefit of renal denervation. Any suitable number of groups or categories are contemplated. For example, the groups may include groups respectively for those patients with low, moderate, and/or high likelihood of therapeutic benefit from renal denervation, based on the PWV. Based on the stratification or classification, the system 100 can recommend the degree to which one or more patients are suitable candidates for renal denervation.

The vessel 80 may represent fluid-filled or surrounded structures, both natural and man-made. The vessel 80 may be within a body of a patient. The vessel 80 may be a blood vessel, such as an artery or a vein of a patient's vascular system, including cardiac vasculature, peripheral vasculature, neural vasculature, renal vasculature, and/or any other suitable lumen inside the body. For example, the intravascular device 110 may be used to examine any number of anatomical locations and tissue types, including without limitation, organs including the liver, heart, kidneys, gall bladder, pancreas, lungs; ducts; intestines; nervous system structures including the brain, dural sac, spinal cord and peripheral nerves; the urinary tract; as well as valves within the heart, chambers or other parts of the heart, and/or other systems of the body. In addition to natural structures, the device intravascular 110 may be used to examine man-made structures such as, but without limitation, heart valves, stents, shunts, filters and other devices. Walls of the vessel 80 define a lumen 82 through which fluid flows within the vessel 80.

The vessel 80 may be located within a body portion. When the vessel 80 is the renal artery, the patient body portion may include the abdomen. Generally, vessel 80 may be located within any portion of the patient body, including the head, neck, chest, abdomen, arms, groin, legs, etc.

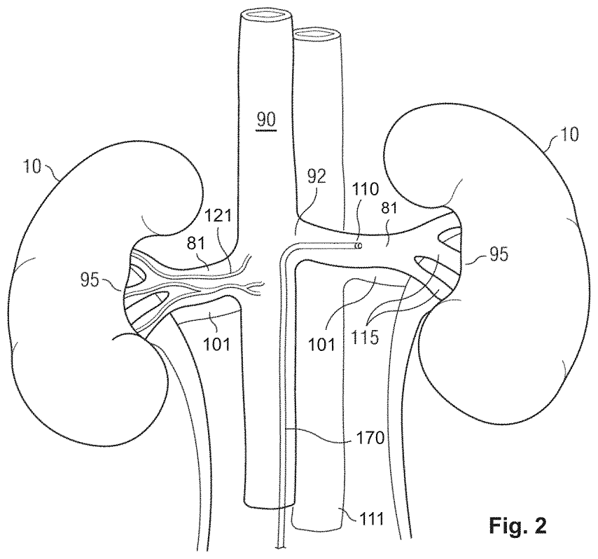

In some embodiments, the intravascular device 110 may include a flexible elongate member 170 such as a catheter, guide wire, or guide catheter, or other long, thin, flexible structure that may be inserted into a vessel 80 of a patient. In some embodiments, the vessel 80 is a renal artery 81 as shown in FIG. 2. While the illustrated embodiments of the intravascular device 110 of the present disclosure have a cylindrical profile with a circular cross-sectional profile that defines an outer diameter of the intravascular device 110, in other instances, all or a portion of the intravascular device may have other geometric cross-sectional profiles (e.g., oval, rectangular, square, ellipse, etc.) or non-geometric cross-sectional profiles. In some embodiments, the intravascular device 110 may or may not include a lumen extending along all or a portion of its length for receiving and/or guiding other instruments. If the intravascular device 110 includes a lumen, the lumen may be centered or offset with respect to the cross-sectional profile of the intravascular device 110.

The intravascular device 110, or the various components thereof, may be manufactured from a variety of materials, including, by way of non-limiting example, plastics, polytetrafluoroethylene (PTFE), polyether block amide (PEBAX), thermoplastic, polyimide, silicone, elastomer, metals, such as stainless steel, titanium, shape-memory alloys such as Nitinol, and/or other biologically compatible materials. In addition, the intravascular device may be manufactured in a variety of lengths, diameters, dimensions, and shapes, including a catheter, guide wire, a combination of catheter and guide wire, etc. For example, in some embodiments the flexible elongate member 170 may be manufactured of a length ranging from approximately 115 cm-185 cm. In one particular embodiment, the flexible elongate member 170 may be manufactured to have length of approximately 135 cm. In some embodiments, the flexible elongate member 170 may be manufactured to have an outer transverse dimension or diameter ranging from about 0.35 mm-2.67 mm (1 Fr-8 Fr). In one embodiment, the flexible elongate member 170 may be manufactured to have a transverse dimension of 2 mm (6 Fr) or less, thereby permitting the intravascular device 110 to be configured for insertion into the renal vasculature of a patient. These exemplary dimensions are provided for illustrative purposes only and are not intended to be limiting. Generally, the intravascular device 110 is sized and shaped such that it may be moved inside the vasculature (or other internal lumen(s)) of a patient such that the flow and/or pressure and cross-sectional area of a vessel 80 may be monitored from within the vessel 80.

In some embodiments, the intravascular device 110 includes a sensor 204 disposed along the length of the flexible elongate member 170. In one embodiment, the sensor 204 may be disposed at a distal end of the flexible elongate member 170. The sensor 204 may be configured to collect data about conditions within the vessel 80.

In one embodiment, the sensor 204 may be configured to acquire intravascular blood flow data. For example, the sensor 204 may be disposed on a guide wire. The sensor 204 may be an electronic, electromechanical, mechanical, optical, and/or other suitable type of sensor. In some embodiments, the sensor 204 may be configured to measure the velocity of blood flow within a blood vessel of a patient. For example, in an embodiment in which the sensor 204 is a flow sensor, flow data obtained by the sensor 204 can be used to calculate physiological variables such as coronary flow reserve (CFR), vascular flow reserve (vFR), and renal flow reserve (RFR). In some examples, pressure data obtained by a pressure sensor may also be used to calculate a physiological pressure ratio (e.g., FFR, iFR, Pd/Pa, or any other suitable pressure ratio).

In other embodiments, the sensor 204 may be configured to obtain intravascular ultrasound (IVUS) data used to generate IVUS images. In other embodiments, the sensor 204 may be other types of imaging sensors, such as an intracardiac echocardiography (ICE), optical coherence tomography (OCT), or intravascular photoacoustic (IVPA) imaging sensor. In an example, the imaging sensor can include one or more ultrasound transducer elements, including an array of ultrasound transducer elements.

In another embodiment, the sensor 204 may be configured to monitor a pressure within the vessel 80. For example, the sensor 204 may periodically measure the pressure of fluid (e.g., blood) at the location of the sensor 204 inside the vessel 80. In an example, the sensor 204 may be a capacitive pressure sensor, or in particular, a capacitive MEMS pressure sensor. In another example, the sensor 204 may be a piezo-resistive pressure sensor. In another example, the sensor 204 may be an optical pressure sensor. In some instances, the sensor 204 may include components similar or identical to those found in commercially available pressure monitoring elements such as the PrimeWire PRESTIGE® pressure guide wire, the PrimeWire® pressure guide wire, and the ComboWire® XT pressure and flow guide wire, each available from Volcano Corporation. In some instances, the sensor 204 may include components similar or identical to the OmniWire pressure guide wire, Verrata pressure guide wire, and/or the Verrata Plus available from Koninklijke Philips N.V. In some embodiments, blood pressure measurements may be used to identify and/or quantify pulse waves passing through the vessel.

The sensor 204 may be contained within the body of the intravascular device 110. The sensor 204 may be disposed circumferentially around a distal portion of the intravascular device 110. In other embodiments, the sensor 204 is disposed linearly along the intravascular device 110. The sensor 204 may include one or more transducer elements. The sensor 204 may be movable along a length of the intravascular device 110 and/or fixed in a stationary position along the length of the intravascular device 110. The sensor 204 may be part of a planar or otherwise suitably-shaped array of sensors of the intravascular device 110. In some embodiments, the outer diameter of the flexible elongate member 170 is equal to or larger than the outer diameter of the sensor 204. In some embodiments, the outer diameter of the flexible elongate member and sensor 204 are equal to or less than about 1 mm, which may help to minimize the effect of the intravascular device 110 on flow and/or pressure measurements within the vessel 80. In particular, since a renal artery generally has a diameter of approximately 5 mm, a 1 mm outer diameter of the intravascular device 110 may obstruct less than 4% of the vessel. In some embodiments, a guide wire can at least partially extend through and be positioned within a lumen of the catheter such that the catheter and guide wire are coaxial.

The processing system 130 may be in communication with the intravascular device 110. For example, the processing system 130 may communicate with the intravascular device 110, including the sensor 204, through an interface module 120. The processor 140 may include any number of processors and may send commands and receive responses from the intravascular device 110. In some implementations, the processor 140 controls the monitoring of the flow and/or pressure within the vessel 80 by the sensor 204. In particular, the processor 140 may be configured to trigger the activation of the sensor 204 to measure flow and/or pressure at specific times. Data from the sensor 204 may be received by a processor of the processing system 130. In other embodiments, the processor 140 is physically separated from the intravascular device 110 but in communication with the intravascular device 110 (e.g., via wireless communications). In some embodiments, the processor is configured to control the sensor 204.

The processor 140 may include an integrated circuit with power, input, and output pins capable of performing logic functions such as commanding the sensor 204 and receiving and processing data. The processor 140 may include any one or more of a microprocessor, a controller, a digital signal processor (DSP), an application specific integrated circuit (ASIC), a field-programmable gate array (FPGA), or equivalent discrete or integrated logic circuitry. In some examples, processor 140 may include multiple components, such as any combination of one or more microprocessors, one or more controllers, one or more DSPs, one or more ASICs, or one or more FPGAs, as well as other discrete or integrated logic circuitry. The functions attributed to the processor 140 herein may be embodied as software, firmware, hardware or any combination thereof.

The processing system 130 may include one or more processors or programmable processor units running programmable code instructions for implementing the pulse wave velocity determination methods described herein, among other functions. The processing system 130 may be integrated within a computer and/or other types of processor-based devices. For example, the processing system 130 may be part of a console, tablet, laptop, handheld device, or other controller used to generate control signals to control or direct the operation of the intravascular device 110. In some embodiments, a user may program or direct the operation of the intravascular device 110 and/or control aspects of the display 160. In some embodiments, the processing system 130 may be in direct communication with the intravascular device 110 (e.g., without an interface module 120), including via wired and/or wireless communication techniques.

Moreover, in some embodiments, the interface module 120 and processing system 130 are collocated and/or part of the same system, unit, chassis, or module. Together the interface module 120 and processing system 130 assemble, process, and render the sensor data for display as an image on a display 160. For example, in various embodiments, the interface module 120 and/or processing system 130 generate control signals to configure the sensor 204, generate signals to activate the sensor 204, perform calculations of sensor data, perform amplification, filtering, and/or aggregating of sensor data, and format the sensor data as an image for display. The allocation of these tasks and others may be distributed in various ways between the interface module 120 and processing system 130. In particular, the processing system 130 may use the received intravascular data to calculate a pulse wave velocity of the fluid (e.g., blood) inside the vessel 80. The interface module 120 can include circuitry configured to facilitate transmission of control signals from the processing system 130 to the intravascular device 110, as well as the transmission of intravascular data from the intravascular device 110 to the processing system 130. In some embodiments, the interface module 120 can provide power to the sensor 204. In some embodiments, the interface module can perform signal conditioning and/or pre-processing of the intravascular data prior to transmission to the processing system 130.

The processing system 130 may be in communication with an electrocardiograph (ECG) console configured to obtain ECG data from electrodes positioned on the patient. For example, ECG system electrodes may be positioned on the skin of the patient body. ECG signals are representative of electrical activity of the heart and can be used to identify the patient's cardiac cycle and/or portions thereof. In some instances, the processing system 130 can utilize different formulas to calculate PWV based on whether the intravascular data obtained by the intravascular device 110 is obtained over an entire cardiac cycle and/or a portion thereof. The ECG data can be used to identify the beginning and ending of the previous, current, and next cardiac cycle(s), the beginning and ending of systole, the beginning and ending of diastole, among other portions of the cardiac cycle. Generally, one or more identifiable features of the ECG signal (including without limitation, the start of a P-wave, the peak of a P-wave, the end of a P-wave, a PR interval, a PR segment, the beginning of a QRS complex, the start of an R-wave, the peak of an R-wave, the end of an R-wave, the end of a QRS complex (J-point), an ST segment, the start of a T-wave, the peak of a T-wave, and the end of a T-wave) can be utilized to select relevant portions of the cardiac cycle. The ECG console may include features similar or identical to those found in commercially available ECG elements such as the PageWriter cardiograph system available from Koninklijke Philips N.V.

Various peripheral devices may enable or improve input and output functionality of the processing system 130. Such peripheral devices may include, but are not necessarily limited to, standard input devices (such as a mouse, joystick, keyboard, etc.), standard output devices (such as a printer, speakers, a projector, graphical display screens, etc.), a CD-ROM drive, a flash drive, a network connection, and electrical connections between the processing system 130 and other components of the intravascular system 100. By way of non-limiting example, the processing system 130 may manipulate signals from the intravascular device 110 to generate an image on the display 160 representative of the acquired flow data, pressure data, imaging data, PWV calculations, and/or combinations thereof. Such peripheral devices may also be used for downloading software containing processor instructions to enable general operation of the intravascular device 110 and/or the processing system 130, and for downloading software implemented programs to perform operations to control, for example, the operation of any auxiliary devices coupled to the intravascular device 110. In some embodiments, the processing system 130 may include a plurality of processing units employed in a wide range of centralized or remotely distributed data processing schemes.

The memory 150 may be a semiconductor memory such as, for example, read-only memory, a random access memory, a FRAM, or a NAND flash memory. The memory 150 may interface with the processor 140 and associated processors such that the processor 140 may write to and read from the memory 150. For example, the processor 140 may be configured to receive data from the intravascular device 110 and/or the interface module 120 and write that data to the memory 150. In this manner, a series of data readings may be stored in the memory 150. The processor 140 may be capable of performing other basic memory functions, such as erasing or overwriting the memory 150, detecting when the memory 150 is full, and other common functions associated with managing semiconductor memory.

FIG. 2 is a diagrammatic view of an intravascular device positioned within the renal artery of a patient, according to aspects of the present disclosure. FIG. 2 illustrates the intravascular device 110 of FIG. 1 disposed within the human renal anatomy. The human renal anatomy includes kidneys 10 that are supplied with oxygenated blood by right and left renal arteries 81, which branch off an abdominal aorta 90 at the renal ostia 92 to enter the hilum 95 of the kidney 10. The abdominal aorta 90 connects the renal arteries 81 to the heart (not shown). Deoxygenated blood flows from the kidneys 10 to the heart via renal veins 101 and an inferior vena cava 111.

Left and right renal plexi or nerves 121 surround the left and right renal arteries 81, respectively. Anatomically, the renal nerve 121 forms one or more plexi within the adventitial tissue surrounding the renal artery 81. For the purpose of this disclosure, the renal nerve is defined as any individual nerve or plexus of nerves and ganglia that conducts a nerve signal to and/or from the kidney 10 and is anatomically located on the surface of the renal artery 81, parts of the abdominal aorta 90 where the renal artery 81 branches off the aorta 90, and/or on inferior branches of the renal artery 81. Nerve fibers contributing to the plexi arise from the celiac ganglion, the lowest splanchnic nerve, the corticorenal ganglion, and the aortic plexus. The renal nerves 121 extend in intimate association with the respective renal arteries into the substance of the respective kidneys 10. The nerves are distributed with branches of the renal artery to vessels of the kidney 10, the glomeruli, and the tubules. Each renal nerve 221 generally enters each respective kidney 10 in the area of the hilum 95 of the kidney, but may enter the kidney 10 in any location, including the location where the renal artery 81, or a branch of the renal artery 81, enters the kidney 10.

Additionally displayed in FIG. 2 is the intravascular device 110 described with reference to FIG. 1. The flexible elongate member 170 of the intravascular device 110 is shown extending through the abdominal aorta and into the left renal artery 81. In alternate embodiments, intravascular device 110 may be sized and configured to travel through the inferior renal vessels 115 as well. Specifically, the intravascular device 110 is shown extending through the abdominal aorta and into the left renal artery 81. In alternate embodiments, the catheter may be sized and configured to travel through the inferior renal vessels 115 as well.

Proper renal function is essential to maintenance of cardiovascular homeostasis so as to avoid hypertensive conditions. Excretion of sodium is key to maintaining appropriate extracellular fluid volume and blood volume, and ultimately controlling the effects of these volumes on arterial pressure. Under steady-state conditions, arterial pressure rises to that pressure level which results in a balance between urinary output and water and sodium intake. If abnormal kidney function causes excessive renal sodium and water retention, as occurs with sympathetic overstimulation of the kidneys through the renal nerves 121, arterial pressure will increase to a level to maintain sodium output equal to intake. In hypertensive patients, the balance between sodium intake and output is achieved at the expense of an elevated arterial pressure in part as a result of the sympathetic stimulation of the kidneys through the renal nerves 121. Renal denervation may help alleviate the symptoms and sequelae of hypertension by blocking or suppressing the efferent and afferent sympathetic activity of the kidneys 10.

In some embodiments, the vessel 80 in FIG. 1 may be a renal vessel consistent with the arteries 81 of FIG. 2 and the pulse wave velocity is determined in the renal artery. The processing system 130 may determine the pulse wave velocity (PWV) in the renal artery. The processing system 130 may determine a renal denervation therapy recommendation based on the pulse wave velocity in a renal artery. For example, patients that are more likely or less likely to benefit therapeutically from renal denervation may be selected based on PWV measurements. In that regard, based at least on the PWV of blood in the renal vessel, the processing system 130 can perform patient stratification for renal denervation.

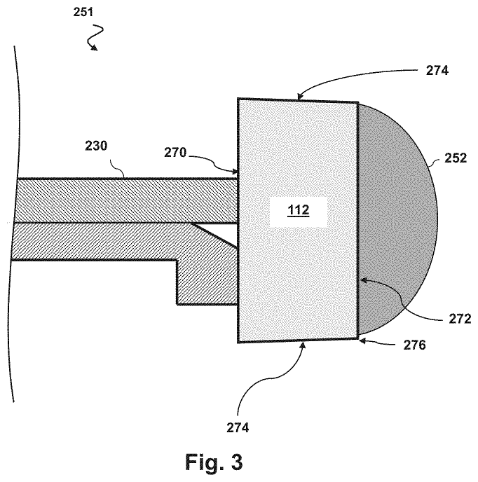

FIG. 3 is a diagrammatic cross-sectional view of an example sensor assembly 251, which may for example be included in the intravascular device of FIG. 1. More specifically, FIG. 3 illustrates a sensor assembly 251 that includes a sensing component 112 and an acoustic matching layer 252. All or a portion of the sensing component 112 and/or the acoustic matching layer 252 can be positioned within a housing. As indicated by the position of the sensing component 204 illustrated in FIG. 1, the sensor assembly 251 may be included in a distal portion of the intravascular device 102 such that the surface 272 of the sensing component 112 faces distally.

As illustrated in FIG. 3, the sensing component 112 includes a proximal surface 270, an opposite, distal surface 272, and a side surface 274. In some embodiments, one or more of the proximal surface 270, the distal surface 272, or the side surface 274 may be coated in an insulating layer 276. The insulating layer 276 may be formed from parylene, which may be deposited on the one or more surfaces, for example. The insulating layer 276 may additionally or alternatively be formed from any other suitable insulating material. In some embodiments, the insulating layer 276 may prevent a short (e.g., an electrical failure), which may otherwise be caused by contact between a conductive portion of the sensing component 112 and the housing, which may be formed with a metal and at least partially surrounds the sensing component 112 (e.g., the sides of sensing component 112). As used herein, references to the distal surface 272 encompass the insulating layer 276 in embodiments where a distal end of the sensing component 112 is covered by the insulating layer 276, references to the proximal surface 270 encompass the insulating layer in embodiments where a proximal end of the sensing component 112 is covered by the insulating layer 276, and references to the side surface 274 encompass the insulating layer in embodiments where the side of the sensing component 112 is covered by the insulating layer 276 unless indicated otherwise.

In some embodiments, the sensing component 112 may include a transducer element, such as an ultrasound transducer element on the distal surface 272 such that the transducer element faces distally and may be used by the sensing component 112 to obtain sensor data corresponding to a structure distal of the sensing component 112. The sensing component 112 may additionally or alternatively include a transducer element on the proximal surface 270 such that the transducer faces proximally and may be used to obtain sensor data corresponding to a structure proximal of the sensing component. A transducer element may additionally or alternatively be positioned on a side surface 274 (e.g., on a perimeter or circumference) of the sensing component 112 in some embodiments.

As further illustrated, the sensing component 112 is coupled to the multi-filar conductor bundle 230. At least a portion (e.g., a distal portion) of the multi-filar conductor bundle 230 can extend through the housing in which the sensing component 112 is positioned. In some embodiments, the multi-filar conductor bundle 230 and the sensing component 112 may be physically (e.g., mechanically) coupled. Further, one or more filars (e.g., conductive members) of the multi-filar conductor bundle 230 may electrically couple to (e.g., be in electrical communication) with the sensing component 112. In particular, one or more filars of the multi-filar conductor bundle 230 may couple to an element, such as a transducer (e.g., an ultrasound transducer), of the sensing component 112 and may provide power, control signals, an electrical ground or signal return, and/or the like to the element. As described above, such an element may be positioned on the distal surface 272 of the sensor. In that regard, in some embodiments, one or more filars of the multi-filar conductor bundle 230 may extend through a cutout or hole in the sensing component 112 (e.g., in at least the proximal surface 270) to establish electrical communication with an element on the distal surface 272 of the sensor. Filars may additionally or alternatively wrap around the side surface 274 to establish electrical communication with the element on the distal surface 272. Moreover, in some embodiments, filars of the multi-filar conductor bundle 230 may terminate at and/or electrically couple to the proximal surface 270 (e.g., to an element on the proximal surface 270) of the sensing component 112. Further, in some embodiments, a subset of the filars of the multi-filar conductor bundle 230 may extend to the distal surface 272 and/or electrically couple to an element at the distal surface 272, while a different subset of the filars may electrically couple to an element at the proximal surface 270, for example.

In some embodiments, the multi-filar conductor bundle 230 may be coated in the insulating layer 276. In some embodiments, for example, the multi-filar conductor bundle 230 and the sensing component 112 may be coupled together in a sub-assembly before being positioned in the housing. In such embodiments, the insulating layer 276 may be applied (e.g., coated and/or deposited) onto the entire sub-assembly, resulting in an insulating layer 276 on both the sensing component 112 and the multi-filar conductor bundle 230.

In some embodiments, the acoustic matching layer 252 may be positioned on (e.g., over) the distal surface 272 of the sensing component 112. In particular, the acoustic matching layer 252 may be disposed directly on the sensing component 112, or the acoustic matching layer 252 may be disposed on the insulating layer 276 coating the sensing component 112. Further, the acoustic matching layer 252 may be disposed on a transducer element (e.g., an ultrasound transducer element) positioned on the sensing component (e.g., the distal surface 272) and/or at least a portion of a conductive filar of the multi-filar conductor bundle 230 that is in communication with the transducer element, such as a filar extending through a hole or along a side of the sensing component 112. To that end, the acoustic matching layer 252 may contact and/or at least partially surround the portion of the conductive filar and/or the transducer element. Moreover, the acoustic matching layer 252 may provide acoustic matching to the sensing component 112 (e.g., to an ultrasound transducer of the sensing component 112). For instance, the acoustic matching layer 252 may minimize acoustic impedance mismatch between the ultrasound transducer and a sensed medium, such as a fluid and/or a lumen that the intravascular device 102 is positioned within. In that regard, the acoustic matching layer 252 may be formed from any suitable material, such as a polymer or an adhesive, to provide acoustic matching with the sensing component 112. The portion of the acoustic matching layer 252 positioned on the distal surface 272 may include and/or be formed from the same material as a portion of the acoustic matching layer positioned on the side surface 274 and/or the proximal surface 270. Further, the acoustic matching layer 252 may be applied to the sensing component 112 before or after the sensing component 112 is positioned within the housing during assembly of the sensor assembly 251. In this regard, the portion of the acoustic matching layer 252 positioned on the distal surface 272 and the portion of the acoustic matching layer positioned on the side surface 274 and/or the proximal surface 270 may be included in the sensor assembly 251 in the same or different steps. Further, in addition to the one or more materials the acoustic matching layer 252 is formed from, the acoustic matching layer 252 may provide acoustic matching with the sensing component 112 via one or more dimensions of the acoustic matching layer 252.

In some embodiments, the sensor assembly 251 may include an atraumatic distal tip. In some embodiments, the distal tip may include the same material as the acoustic matching layer 252. In some embodiments, the distal tip may include a different material than the acoustic matching layer 252. Additionally or alternatively the distal tip may be formed from one or more layers of materials. The layers may include different materials and/or different configurations (e.g., shape and/or profile, thickness, and/or the like). Further, the distal tip may be arranged to cover the distal surface 272 of the sensing component 112. In some embodiments, the distal tip may also cover a distal end 272 of the housing in which the sensing component 112 is at least partially positioned. Moreover, while the distal tip may be of a domed shape, embodiments are not limited thereto. In this regard, the distal tip may include a flattened profile or any suitable shape. In some embodiments, the entire sensing component 112 may be positioned within (e.g., surrounded by the continuous surface of) the housing.

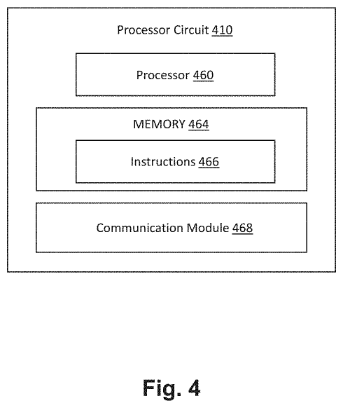

FIG. 4 is a schematic diagram of a processor circuit, according to aspects of the present disclosure. The processor circuit 410 may be implemented in the processing system 130 of FIG. 1. In an example, the processor circuit 410 may be in communication with the intraluminal imaging device 110 and/or the display 160 within the system 100. The processor circuit 410 may include a processor and/or communication interface. One or more processor circuits 410 are configured to execute the operations described herein. As shown, the processor circuit 410 may include a processor 460, a memory 464, and a communication module 468. These elements may be in direct or indirect communication with each other, for example via one or more buses.

The processor 460 may include a CPU, a GPU, a DSP, an application-specific integrated circuit (ASIC), a controller, an FPGA, another hardware device, a firmware device, or any combination thereof configured to perform the operations described herein. The processor 460 may also be implemented as a combination of computing devices, e.g., a combination of a DSP and a microprocessor, a plurality of microprocessors, one or more microprocessors in conjunction with a DSP core, or any other such configuration.

The memory 464 may include a cache memory (e.g., a cache memory of the processor 460), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 464 includes a non-transitory computer-readable medium. The memory 464 may store instructions 466. The instructions 466 may include instructions that, when executed by the processor 460, cause the processor 460 to perform the operations described herein with reference to the device 110 and/or the processing system 130 (FIG. 1). Instructions 466 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 468 can include any electronic circuitry and/or logic circuitry to facilitate direct or indirect communication of data between the processor circuit 410, the device 110, and/or the display 160. In that regard, the communication module 468 can be an input/output (I/O) device. In some instances, the communication module 468 facilitates direct or indirect communication between various elements of the processor circuit 410 and/or the device 110 (FIG. 1) and/or the processing system 130 (FIG. 1).

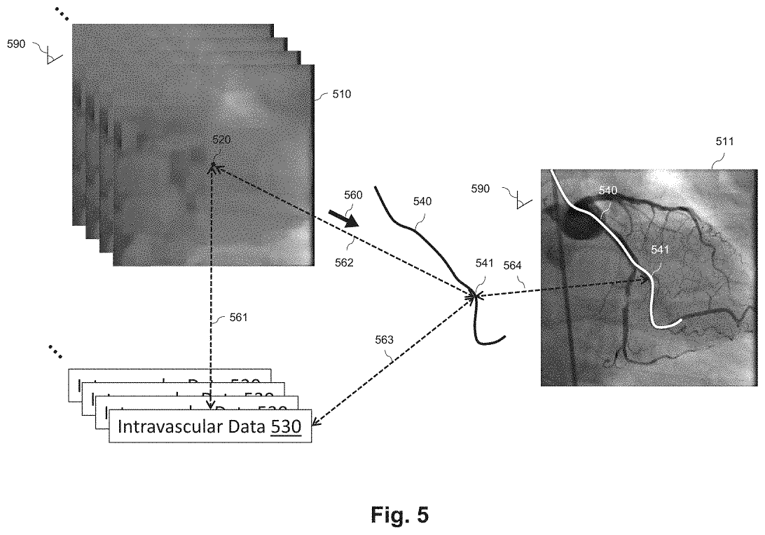

FIG. 5 is a diagrammatic view of a relationship between x-ray fluoroscopy images 510, intravascular data 530, and a path 540 defined by the motion of an intravascular device, according to aspects of the present disclosure. FIG. 5 describes a method of coregistering intravascular data 530 including intravascular images with corresponding locations on one or more fluoroscopy images 510 of the same region of a patient's anatomy. The steps, principles, and/or methods described with reference to FIG. 5 may be described as a coregistration process. A coregistration process may alternatively be referred to as a coregistration procedure. A coregistration process may be performed by a processor circuit of the system (ie, the processor circuit 410). By performing the coregistration process described in FIG. 5, the processor circuit may determine the position or location at which any data was received and determine distances between any locations within the patient anatomy.

The patient anatomy may be imaged with an x-ray device while a physician performs a pullback with an intravascular device 520, e.g., while the intravascular device 520 moves through a blood vessel of the anatomy. The intravascular device may be substantially similar to the intravascular device described with reference to FIG. 1. In some embodiments, the fluoroscopy images 510 may be obtained while no contrast agent is present within the patient vasculature. Such an embodiment is shown by the fluoroscopy images 510 in FIG. 5. The radiopaque portion of the intravascular device 520 is visible within the fluoroscopy image 510. The fluoroscopy images 510 may correspond to a continuous image stream of fluoroscopy images and may be obtained as the patient anatomy is exposed to a reduced dose of x-radiation. It is noted that the fluoroscopy images 510 may be acquired with the x-ray source and the x-ray detector positioned at any suitable angle in relation to the patient anatomy. This angle is shown by angle 590.

The intravascular device 520 may be any suitable intravascular device. As the intravascular device 520 moves through the patient vasculature, the x-ray imaging system may acquire multiple fluoroscopy images 510 showing the radiopaque portion of the intravascular device 520. In this way, each fluoroscopy image 510 shown in FIG. 5 may depict the intravascular device 520 positioned at a different location such that a processor circuit may track the position of the intravascular device 520 over time.

As the intravascular device 520 is pulled through the patient vasculature, it may acquire intravascular data 530. In an example, the intravascular data 530 shown in FIG. 5 may be IVUS images. However, the intravascular data may be any suitable data, including IVUS images, pressure and flow data, OCT images, intravascular photoacoustic (IVPA) images, or any other measurements or metrics relating to blood pressure, blood flow, lumen structure, or other physiological data acquired during a pullback of an intravascular device.

As the physician pulls the intravascular device 520 through the patient vasculature, each intravascular data point 530 acquired by the intravascular device 520 may be associated with a position within the patient anatomy in the fluoroscopy images 510, as indicated by the arrow 561. For example, the first IVUS image 530 shown in FIG. 4 may be associated with the first fluoroscopy image 510. The first IVUS image 530 may be an image acquired by the intravascular device 520 at a position within the vasculature, as depicted in the first fluoroscopy image 510 as shown by the intravascular device 520 within the image 510. Similarly, an additional IVUS image 530 may be associated with an additional fluoroscopy image 510 showing the intravascular device 520 at a new location within the image 510, and so on. The processor circuit may determine the locations of the intravascular device 520 within each acquired x-ray image 510 by any suitable method. For example, the processor circuit may perform various image processing techniques, such as edge identification of the radiopaque marker, pixel-by-pixel analysis to determine transition between light pixels and dark pixels, filtering, or any other suitable techniques to determine the location of the imaging device 520. In some embodiments, the processor circuit may use various artificial intelligence methods including deep learning techniques such as neural networks or any other suitable techniques to identify the locations of the imaging device 520 within the x-ray images 510.

Any suitable number of IVUS images or other intravascular data points 530 may be acquired during an intravascular device pullback and any suitable number of fluoroscopy images 510 may be obtained. In some embodiments, there may be a one-to-one ratio of fluoroscopy images 510 and intravascular data 530. In other embodiments, there may be differing numbers of fluoroscopy images 510 and/or intravascular data 530. The process of co-registering the intravascular data 530 with one or more x-ray images may include some features similar to those described in U.S. Pat. No. 7,930,014, titled, “VASCULAR IMAGE CO-REGISTRATION,” and filed Jan. 11, 2006, which is hereby incorporated by reference in its entirety. The co-registration process may also include some features similar to those described in U.S. Pat. Nos. 8,290,228, 8,563,007, 8,670,603, 8,693,756, 8,781,193, 8,855,744, and 10,076,301, all of which are also hereby incorporated by reference in their entirety.

The system 100 may additionally generate a fluoroscopy-based 2D pathway 540 defined by the positions of the intravascular device 520 within the x-ray fluoroscopy images 510. The different positions of the intravascular device 520 during pullback, as shown in the fluoroscopy images 510, may define a two-dimensional pathway 540, as shown by the arrow 560. The fluoroscopy-based 2D pathway 540 reflects the path of one or more radiopaque portions of the intravascular device 520 as it moved through the patient vasculature as observed from the angle 590 by the x-ray imaging device. The fluoroscopy-based 2D pathway 540 defines the path as measured by the x-ray device which acquired the fluoroscopy images 510, and therefore shows the path from the same angle 590 at which the fluoroscopy images were acquired. Stated differently, the 2D pathway 540 describes the projection of the 3D path followed by the device onto the imaging plane at the imaging angle 590. In some embodiments, the pathway 540 may be determined by an average of the detected locations of the intravascular device 520 in the fluoroscopy images 510. For example, the pathway 540 may not coincide exactly with the guidewire in any fluoroscopy image 510 selected for presentation.

As shown by the arrow 562, because the two-dimensional path 540 is generated based on the fluoroscopy images 510, each position along the two-dimensional path 540 may be associated with one or more fluoroscopy images 510. As an example, at a location 541 along the path 540, the first fluoroscopy image 510 may depict the intravascular device 520 at that same location 541. In addition, because a correspondence was also established between the fluoroscopy images 510 and the intravascular data 530 as shown by the arrow 561, intravascular data 530, such as the first IVUS image shown, may also be associated with the location 541 along the path 540 as shown by the arrow 563.

Finally, the path 540 generated based on the locations of the intravascular device 520 within the fluoroscopy images 510 may be overlaid onto any suitable fluoroscopy image 511 (e.g., one of the fluoroscopic images 510 in the fluoroscopic image stream). In this way, any location along the path 540 displayed on the fluoroscopy image 511 may be associated with IVUS data such as an IVUS image 530, as shown by the arrow 564. For example, IVUS image 530 shown in FIG. 4 may be acquired simultaneously with the fluoroscopy image 510 shown and the two may be associated with each other as shown by the arrow 561. The fluoroscopy image 510 may then indicate the location of the intravascular device 520 along the path 540, as shown by the arrow 562, thus associating the IVUS image 530 with the location 541 along the path 540 as shown by the arrow 563. Finally, the IVUS image 530 may be associated with the location within the fluoroscopy image 510 at which it was acquired by overlaying the path 540 with associated data on the fluoroscopy image 511. The pathway 540 itself may or may not be displayed on the image 511.

In the illustrated embodiment of FIG. 5, the co-registered IVUS images are associated with one of the fluoroscopic images obtained without contrast such that that the position at which the IVUS images are obtained is known relative to locations along the guidewire. In other embodiments, the co-registered IVUS images are associated with an x-ray image obtained with contrast (in which the vessel is visible) such that that the position at which the IVUS images are obtained is known relative to locations along the vessel.

In some aspects, extraluminal images may be obtained from more than one angle relative to the patient anatomy. In that regard, the shape and position of the vessel and/or guidewire during the imaging procedure may be known in greater detail. For example, by obtaining two or more extraluminal images from different angles, two or more projections of the path of the device may be obtained. These two or more projections allow the three-dimensional anatomy of the vessel to be determined. In some aspects, a 3D view or model of the vessel can be generated and displayed based on the two or more projections. In some aspects, using the two or more projections to determine the 3D anatomy may improve distance measurement accuracy. In addition, the 3D anatomy may also improve errors relating to foreshortening of the vessel. In some aspects, obtaining the 3D anatomy of the vessel may include techniques or procedures similar to those used for obtaining computed tomography (CT) or magnetic resonance (MR) images acquired pre-intervention.

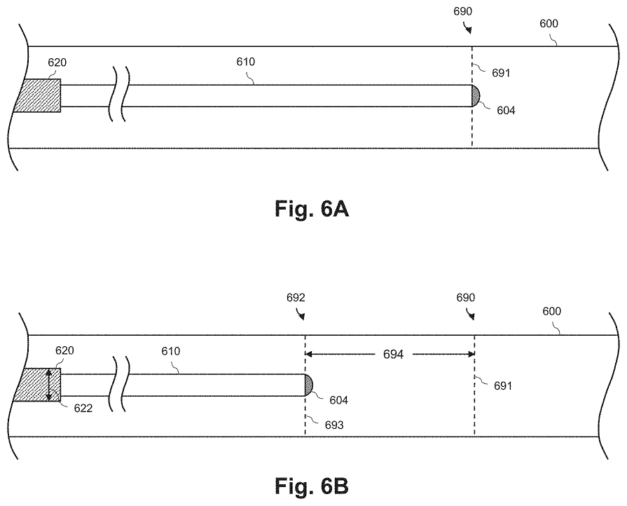

FIG. 6A is a diagrammatic view of an intravascular device 610 within a lumen 600, according to aspects of the present disclosure.

In some examples, the intravascular device 610 may be similar to the device 110 described with reference to FIG. 1. In that regard, the intravascular device 610 may be a blood flow measurement device, a pressure sensing device, an intraluminal imaging device, or any other device. In the example described with reference to FIG. 6A, the device 610 may be a blood flow measurement device.

The lumen 600 shown in FIG. 6A may be any suitable lumen. For example, the lumen 600 may be similar to the vessel 80 described with reference to FIG. 1. In that regard, the lumen 600 may be a body lumen of a patient. In the example described with reference to FIG. 6A, the lumen 600 may be a blood vessel. In some embodiments, the lumen 600 may correspond to a renal artery such as the renal artery 81 described with reference to FIG. 2.

As shown in FIG. 6A, the intravascular device 610 may include various components. For example, in some embodiments, the device 610 may include a guide catheter 620. The device 610 may include a flexible elongate member configured to be positioned within the body lumen of a patient. The flexible elongate member may also be configured to be positioned within the guide catheter 620.

The intravascular device 610 may also include a sensor 604. The sensor 604 may be similar to the sensor 204 described with reference to FIG. 1. In that regard, the sensor 604 may acquire data corresponding to pressure of blood within the lumen 600, flow data relating to the velocity of blood within the lumen 600, intravascular image data of the lumen 600, or any other data. As shown in FIG. 6A, the sensor 604 may be positioned at a distal end of the device 610. In other embodiments, however, the sensor 604 may be positioned at any suitable location along the flexible elongate member, or at any other position of the device 610.

As has been described, during a diagnostic procedure, the intravascular device 610 may be positioned within a vessel (e.g., the lumen 600). A diagnostic procedure may also be referred to as a pullback procedure. In some embodiments, the device 610 may be positioned such that the sensor 604 of the device 610 is positioned at some distal location 690 of the lumen 600. The physician performing the pullback procedure may then direct the system 100 to acquire intravascular data as the device 610 is moved in a proximal direction. In some embodiments, the physician may initially position the intravascular device at a proximal location within the lumen 600 and move the device in a distal direction while acquiring intravascular data.

The position of the device 610 in FIG. 6A may represent the location of the position of the device 610 at a time T1. This time T1 may correspond to an initial phase of a diagnostic procedure or pullback. In some embodiments, the time T1 and corresponding position of the device 610 in FIG. 6A may refer to any time of a pullback procedure. For example, the position of the device 610 in FIG. 6A may illustrate the location of the device 610 at a snapshot of time during a pullback procedure as the device 610 is in motion. As shown in FIG. 6A, the sensor 604 of the device is positioned at a location 690. The location 690 along the lumen 600 is identified by the indicator 691.

FIG. 6B is a diagrammatic view of an intravascular device 610 within a lumen 600, according to aspects of the present disclosure. The device 610 shown in FIG. 6B may be the same device 610 shown in FIG. 5A but at a different time, T2. In this way, a comparison of FIGS. 5A and 5B may illustrate a movement of the device 610 within the lumen 600.

As shown in FIG. 6B, the device 610 may include the same components, as well as any other components, including the guide catheter 620 and the sensor 604. However, as shown in FIG. 6B, the device 610 may have been moved in a proximal direction to a new position. For example, the sensor 604 of the device 610 may be positioned at a location 692 within the lumen 600. The location 692 may be proximal to the location 690. The location 692 may be further identified by an indicator 691.

The location 690 and associated indicator 691 may also be displayed in FIG. 6B. A distance measurement between the locations 690 and 692 may be calculated by the processor circuit 410. This distance measurement may be identified by the indicator 694 in FIG. 6B. The indicator 694 may identify a distance travelled by the device 610 (e.g., the sensor 604 of the device 610) between time T1 of FIG. 5A and time T2 of FIG. 6B. Based on the locations 690 and 692 and the times T1 and T2, a velocity measurement of the device 610 may also be determined.

Also shown in FIG. 6B is an indicator 622. The indicator 622 may identify a diameter of the guide catheter 620. In some embodiments, the guide catheter 620 may be constructed of a radiopaque material. In such an embodiment, because the guide catheter 620 is constructed of a radiopaque material, the guide catheter 620 may be visible within an extraluminal image (e.g., x-ray image obtained without contrast). In addition, the diameter of the guide catheter 620 (shown by indicator 622) may be a known distance measurement. For example, the processor circuit 410 may identify, or receive, a measurement corresponding to the diameter of the guide catheter 620. Processor circuit 410 may be configured to use this measurement (e.g., the diameter of 622) as a reference distance measurement. For example, the processor circuit 410 may be configured to determine a number of pixels associated with the width, or a diameter, of the guide catheter 620. Based on this number of pixels, the processor circuit 410 may determine other distance measurements of features, anatomical structures, devices, or components of devices within an extraluminal image. In one example, a distance measurement corresponding to a length travelled by an intravascular device (e.g., the device 610) may be determined based on the reference distance shown by the diameter of the guide catheter 620. The processor circuit 410 may also be configured to determine distance measurements within an extraluminal image in any other suitable way.

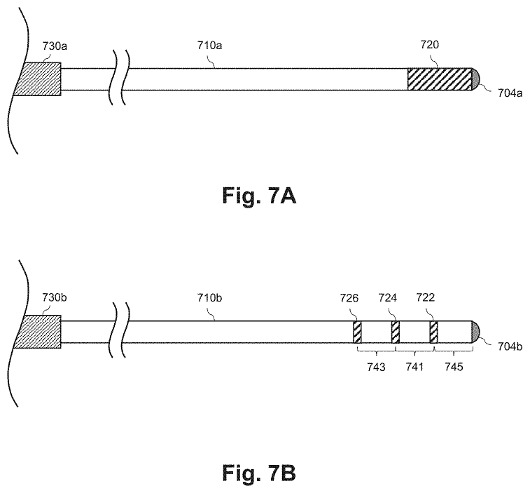

FIG. 7A is a diagrammatic view of an intravascular device 710a according to aspects of the present disclosure.

The intravascular device 710a may be similar to the device 610 described with reference to FIG. 6A and FIG. 6B. The intravascular device 710a may include a guide catheter 730a and a sensor 704a. The intravascular device 710a may additionally include a radiopaque region 720. The radiopaque region 720 may be disposed at any location along the device 710a. In one example, as shown in FIG. 7A, the radiopaque region 720 may be disposed at a distal region of the device 710a.

In some embodiments, like the diameter of the guide catheter 620 of FIG. 6B, the dimensions of the radiopaque region 720 may be known. For example, the length of the region 720 in a longitudinal direction may be known. In some embodiments, the processor circuit 410 may determine or receive a length of the radiopaque region 720. In some embodiments, the processor circuit 410 may receive this length has an input from a user of the system 100. In other embodiments, the processor circuit 410 may automatically determine this length based on the type of device 710a. For example, the processor circuit 410 may receive an input upon bringing the device 710a into communication with an interface module, such as the interface module 120 described with reference to FIG. 1, including a length measurement of the radiopaque region 720.

As described with reference to the diameter of 622 of the guide catheter 620 of FIG. 6B, the known length of the radiopaque region 720 of the device 710a may serve as a reference distance for the processor circuit 410. Specifically, the processor circuit 410 may determine a number of pixels associated with the length of the radiopaque region 720 within an x-ray image obtained without contrast. Based on the number of pixels associated with the known length of the radiopaque region 720, the processor circuit 410 may determine a distance measurement associated with a pixel in an x-ray image. Based on this relationship, the processor circuit 410 may determine a length between any positions of an x-ray image based on the number of pixels separating those positions. For example, a distance traveled by an intravascular device, such as the device 710a, or any of the intravascular devices described in the present disclosure, may be determined based on the number of pixels within an x-ray image, such as a road map image (e.g., the image 511 of FIG. 5), corresponding to the length of travel of the device. In some aspects, the known length of the radiopaque region of the device 710a may also improve distance calculation accuracy. In particular, the known length of the radiopaque region 710a may be compared to an observed length within an extraluminal image to correct errors in distance or length caused by foreshortening, which may be caused by projecting the three-dimensional structure of the vessel or intravascular device onto a two-dimensional image. For example, if the particular length of the radiopaque region of the device 710a is known, but appears within an extraluminal image as shorter than expected, the observed length can be compared to the known length to calculate the degree of foreshortening observed in the particular extraluminal image.

FIG. 7B is a diagrammatic view of an intravascular device 710b according to aspects of the present disclosure.

The intravascular device 710b may be similar to the device 710a described with reference to FIG. 7A and/or the device 610 described with reference to FIG. 6A and FIG. 6B. The intravascular device 710b may similarly include a guide catheter 730b and a sensor 704b. The intravascular device 710b may additionally include one or more radiopaque regions. For example, device 710b may include a radiopaque marker 722, a radiopaque marker 724, and a radiopaque marker 726. These radiopaque markers 722, 724, and 726 may be disposed at any location along the device 710b. In one example, as shown in FIG. 7B, the radiopaque markers 722, 724, and 726 may be disposed at a distal region of the device 710b.

In some embodiments, like the diameter of the guide catheter 620 of FIG. 6B, distance measurements between the radiopaque markers 722, 724, and 726 may be known. Specifically, a length 741 separating the radiopaque marker 722 and 724 may be known. Additionally, a length 743 between the radiopaque marker 724 and the radiopaque marker 726 may be known. In some embodiments, a length 745 between the radiopaque marker 722 and the sensor 704b may be known. In some embodiments, the processor circuit 410 may determine or receive a length of the radiopaque region 720. In some embodiments, the processor circuit 410 may receive these distance measurements (e.g., the length 741, the length 743, and/or the length 745) an input from a user or the device 710b or may automatically determine them, or by any method described with reference to FIG. 7A. These known length measurements may be used by the processor circuit 410 as reference distance measurements as described with reference to FIG. 6A, FIG. 6B, and/or FIG. 7A. In addition, these known length measurements may be used in relation to foreshortening correction as described with reference to FIG. 7A.

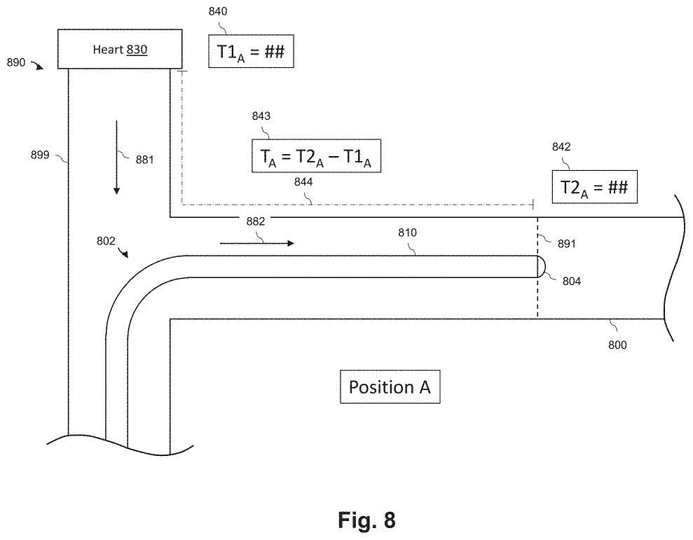

FIG. 8 is a diagrammatic view of an intravascular device within a region of a patient anatomy showing a heart 830, an aorta 899 (e.g., abdominal aorta), and a renal artery 800, according to aspects of the present disclosure.

At a step of the present disclosure, an intravascular device 802 may be positioned within the patient vasculature. Specifically, the device 802 may be positioned such that a sensor 804 of the device is positioned within a renal artery 800 of the patient vasculature at the location 891. For the purposes of this disclosure, this position may correspond to position A, as shown in FIG. 8. In some embodiments, the device 802 may be similar to any of the previously described devices. For example, the device 802 may share characteristics or features of the device shown in FIG. 1, the device of FIG. 3, or the devices described with reference to FIGS. 6A, 6B, 7A, and/or 7B. The device 802 may include a flexible elongate member 810 and a sensor 804. As previously described, the sensor 804 may be a pressure sensor, a blood velocity or blood flow sensor, or an imaging sensor, such as an IVUS imaging assembly. In one example described with reference to FIG. 8, the sensor 804 will be described as a pressure sensor. The device 802 may be sized and shaped so as to be positioned within a body lumen of the patient.

The pressure sensor of the device 802 may be positioned at the location 891 within the renal artery 800. After the sensor 804 is positioned at the location 891, the user of the system, or a processor circuit of the system (e.g., the processor circuit 410), may direct the sensor 804 to begin acquiring intravascular data. As mentioned, this intravascular data may be pressure data, or it may be blood flow data or imaging data. As the sensor 804 acquires intravascular data, the heart 830 may be continuously pumping blood through the patient vasculature. For example, the heart 830 may pump multiple pulses or pulse waves of blood from the heart through the patient vasculature. In the example shown in FIG. 8, a pulse wave from the heart 830 may travel in a downward direction as shown by the arrow 881 and then into the renal artery as shown by the arrow 882. As this pulse wave passes the sensor 804, the sensor 804 may detect a change in pressure of the blood within the renal artery 800.

In some embodiments, the heart 830 of the patient may be monitored by an additional sensor or data acquisition system. For example, the heart 830 of the patient may be monitored by a heart monitor. A heart monitor may include any suitable device or system configured to acquire data relating to the movement of the heart, including individual chambers of the heart, blood pressure data within the heart or at any other location within the patient vasculature, blood flow data, metrics of the vessels of the vasculature including diameter measurements or cross-sectional area measurements, metrics of the chambers of the heart including diameter measurements, cross-sectional area measurements, or volume measurements. In some embodiments, the heart monitor may be an electrocardiogram (ECG) system. In some embodiments, the ECG system may detect a voltage associated with the heart corresponding to the rate at which blood is pumped from the heart. Based on these ECG measurements, a time T1_{A} 840 may be determined to be the time at which a blood pulse wave leaves the heart 830.

Similarly, the pressure data acquired by the sensor 804 may be associated with time data. For example, the time at which the pulse wave which left the heart 830 arrives at the location 891 and is sensed by the pressure sensor 804 may be the time T2_{A} 842 shown in FIG. 8.

Based on the measured time T1_{A} 840 of the pulse wave leaving the heart 830 and the time T2_{A} 842 at which the pulse wave arrived at the sensor 804, the time for the pulse wave to travel from the heart 830 to the location 891 may be determined. This time may be the time T_{A} 843 shown in FIG. 8. The time T_{A} 843 shown in FIG. 8 may be determined by subtracting the time value T1_{A} from the time value T2_{A}. In this way, the time T_{A} is the difference between the time T1_{A} and the time T2_{A}.

In some embodiments, the distance traveled by the pulse wave from the heart 830 to the location 891 corresponding to the pressure sensor 804 may be shown in FIG. 8 by the indicator 844. In some embodiments, the position of the device 802 may be held stationary for a period of time period. For example, the user of the system may ensure that the sensor 804 is stationary within the renal artery 800 at the position 891 for a period of time corresponding to multiple pulse waves from the heart. For example, the user of the system 100 may ensure that the pressure sensor 804 is held stationary at the location 891 for the duration of one, two, three, four, or more pulse waves and or heartbeats of the patient to ensure the accuracy of the data collected.

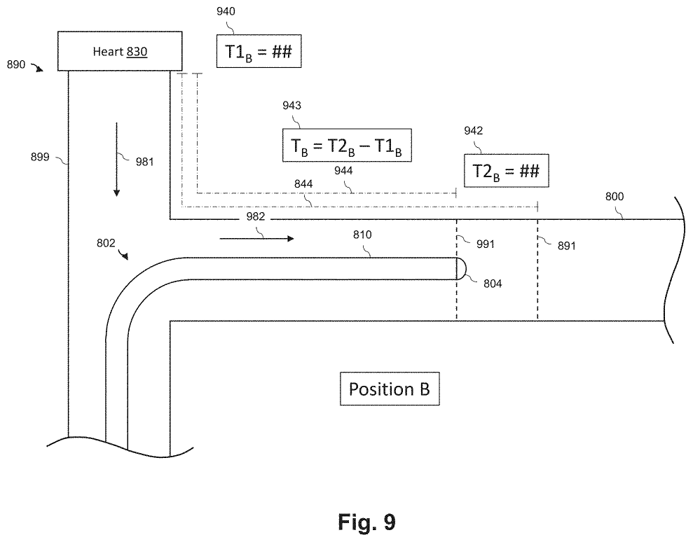

FIG. 9 is a diagrammatic view of an intravascular device within a region of a patient anatomy showing a heart 830, an aorta 899, and a renal artery 800, according to aspects of the present disclosure.

After the time T_{A} 843 is calculated, as described with reference to FIG. 8, the intravascular device 802 may be moved to a different location 991 within the renal artery 800. The location 991 may be some location along the renal artery 800 proximal to the location 891. For example, the pressure data acquired during the presently disclosed procedure may correspond to a pullback procedure. In such an embodiment, the user of this system 100 may position the device 802 at a distal location (e.g., the location 891) and subsequently move the device 802 in a proximal direction. As the device 802 is moved through the patient vasculature, the device 802 may continuously acquire intravascular data. Such an example may be shown in FIG. 9. The position of the device 802 with the sensor 804 at the location 991 may correspond to position B, as shown in FIG. 9.

In some embodiments, the device 802 may be held stationary at the location 991. As previously described with reference to the location 891, the sensor 804 may be held stationary at the location 991 for a period of time corresponding to multiple pulse waves or heartbeats of the patient. In some embodiments, the user of the system 100 may hold the device 802 stationary with the sensor 804 at the position 991 for any length of time, including a length of time associated with the location 891 previously described. In some aspects, a length of time may alternatively be referred to as an amount of time, a duration of time, or any other suitable terms.

As the sensor 804 is held stationary at the location 991, the sensor 804 may be continuously acquiring pressure data. As an example, the heart 830 may send another pulse wave through the patient vasculature as the sensor 804 is positioned at the location 991. For example, and as shown in FIG. 9, the heart 830 may emit a pulse wave which may travel in a downward direction as shown by the arrow 981 and along the renal artery 800 as shown by the arrow 982. Similar to the procedure described with reference to FIG. 8, a time T1_{B} 940 corresponding to the time at which a pulse wave left the heart 830 may be determined. As described previously, this time T1_{B} 940 may be determined based on data received from an ECG system. For example, the ECG system may be in communication with the processor circuit of the system 100. In some embodiments, the ECG system, as well as the intravascular system associated with the device 802, may be in communication with the same processor circuit (e.g., the processor circuit 410).

The pressure sensor 804 may detect a change in pressure as the pulse wave sent from the heart 830 passes through the location 991 within the renal artery 800. As described previously, a time T2_{B} 942 may be calculated. The time T2_{B} 942 may correspond to the time at which the pulse wave which left the heart at time T1_{B} 940 is measured by the pressure sensor 804 at the location 991. The indicator 944 may illustrate the distance traveled by the pulse wave from the heart 830 to the location 991 within the renal artery 800.

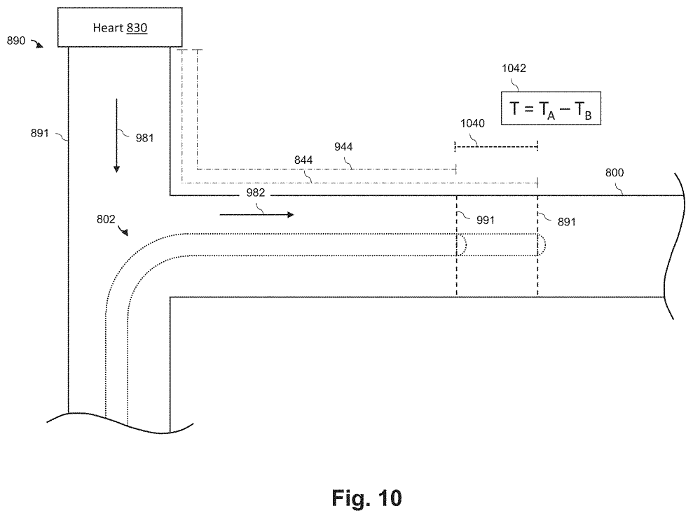

Based on the time T1_{B} 940 and the time T2_{B} 942, a time T_{B} 943 may be calculated. Just as the time T_{A} 843 described with reference to FIG. 8 corresponds to the duration of time it takes for a pulse wave to travel from the heart 830 to location 891, the time T_{B} 943 corresponds to the duration of time it takes for a pulse wave to travel from the heart 830 to the location 991. As a result, as will be described with reference to FIG. 10, the time it takes for a pulse wave to travel from the location 991 to the location 891 may be calculated as a difference between the time T_{A} 843 (FIG. 8) and the time T_{B} 943.

FIG. 10 is a diagrammatic view of an intravascular device within a region of a patient anatomy showing a heart 830, an aorta 899, and a renal artery 800, according to aspects of the present disclosure.

As shown by the time T 1042 and indicator 1040, the distance between the locations 891 and 991 as well as the time for a pulse wave to travel from the location 991 to location 891 may be calculated. Based on these calculations, the pulse wave velocity within the renal artery 800 may also be calculated.

As shown in FIG. 10, the distance 1040 between the location 891 and the location 991 may be calculated. This distance measurement 1040 may be determined based on coregistration data. For example, as described with reference to FIG. 5 previously, because the device (e.g., the device 802) may be moved within the patient vasculature while extraluminal images are obtained of the same region of the patient vasculature, the locations of data acquired by the intravascular device 802 may be known within an extraluminal image. In addition, as described with reference to FIGS. 7A and 7B, radiopaque portions of the intravascular device may provide a reference distance for the processor circuit of the system. Based on these reference distance measurements of radiopaque portions of the device, the processor circuit of the system 100 may determine distance measurements between any positions within an extraluminal image. For example, the location 891 of the device 802 may be determined within an extraluminal image according to any of the coregistration methods described previously. The location 991 of the device 802 may similarly be determined within the same extraluminal image according to the same methods. The distance between these two locations 891 and 991, may then be calculated. This distance may be stored and or displayed as the distance 1040. As shown, the distance 1040 may be a difference between the distance 844 and the distance 944.

Also shown in FIG. 10, a time T 1042 may be displayed. The time T 1042 may be a difference between the time T_{A} 843 (FIG. 8) and the time T_{B} 943 (FIG. 9). It is noted that the time T_{A} and the time T_{B} may vary between different patients, depending on the anatomy of the patient. For example, the times T_{A} or T_{B} may depend on the distance 844 and/or the distance 944 respectively which may vary between different patients and which may not be known during a pulse wave velocity calculation procedure such as the one described herein. The times T_{A} and T_{B} may also depend on various attributes of the patient vasculature, including the elasticity of vessels within the patient, attributes of the heart 830, or any other characteristics of the patient. However, because the difference between the times T_{A} and T_{B} is determined, any errors in the time T_{A} or T_{B}, whether known or otherwise, are not present in the time T 1042.

The pulse wave velocity may refer to the velocity of a blood pulse travelling through a vessel. In this way, the units of pulse wave velocity may be units of velocity, or distance over time. In this case, pulse wave velocity within the renal artery 800 may be determined by dividing the distance 1040 by the time 1042. Pulse wave velocity may be calculated and or displayed to a user with any suitable units of measurement. For example, the pulse wave velocity may be calculated and or displayed as a unit of meters per second, millimeters per second, or by any other unit of velocity measurement.

Aspects of the present disclosure advantageously allows the pulse wave velocity of a blood pulse moving through the renal artery to be calculated in an advantageous manner. In particular, only one sensor of an intravascular device (e.g., the pressure sensor 804, a flow sensor, or an IVUS imaging assembly) is required. Previous methods of calculating pulse wave velocity often require a user to position two intravascular sensors within the renal artery, the two sensors being positioned at a fixed, known distance from one another. By requiring only one sensor, the present disclosure allows physicians to measure pulse wave velocity with a wider range of devices, including devices with only a single sensor. The single sensor obtains intravascular data at least two locations of the blood vessel as a result of the intravascular device moving within the vessel (e.g., a pullback). The intravascular data obtained by the single sensor is co-registered to an extraluminal image (e.g., an x-ray image). The distance between the two locations where the single sensor obtained the intravascular data can be determined based on the co-registration, and this distance can be used in the PWV calculation. The time used in the PWV calculation can be determined based on one or more identifiable feature in a waveform of the intravascular data obtained by the single sensor and one or more identifiable features in a waveform of physiological data obtained from another physiological sensor (e.g., ECG, another pressure sensor, etc.)

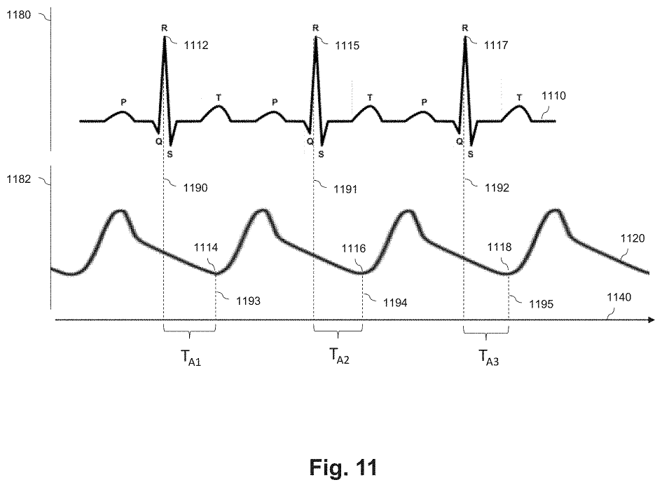

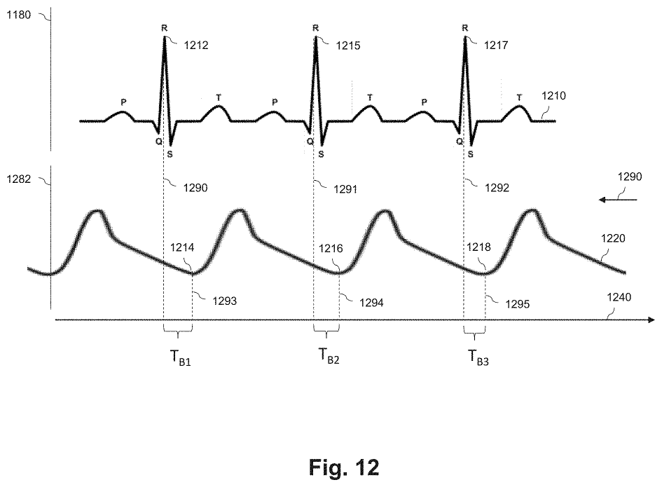

FIG. 11 is a diagrammatic view of an ECG curve 1110 and a blood pressure curve 1120 associated with a time axis 1140 and acquired before an intravascular device is moved within a renal artery, according to aspects of the present disclosure. FIGS. 8-10 describe previously, illustrate how a time value, T, which corresponds to the amount of time it takes for a blood pulse wave to travel from the sensor at location 991 to the sensor at location 891, is calculated. This description is in regards to a single blood pulse cycle. This calculation may be made more accurate by acquiring time values over multiple cycles as shown and described with reference to FIGS. 11-12. Thus, multiple time values T_{A1}, T_{A2}, etc. are acquired with the probe at position A (FIG. 8) and multiple time values T_{B1}, T_{B2}, etc. are acquired with the probe at position B (FIG. 9). These may be combined (e.g., averaged, or otherwise combined) to result in the time T 1042.

FIG. 11 may illustrate two plots of data corresponding to the measurements and calculations described with reference to FIG. 8. As an example, the ECG system described with reference to FIGS. 8-10 may acquire data such as the ECG curve 1110 shown in FIG. 11. The ECG data and intravascular pressure data shown in FIG. 11 may be obtained simultaneously. In that regard, the processor circuit of the system 100 may be configured to synchronize intravascular data, including pressure, flow, etc., with ECG data. The ECG curve 1110 may correspond to the movement of the heart 830 (FIGS. 8-10) during a pulse wave velocity measurement procedure. In this way, the ECG curve 1110 may be described as a cyclic waveform. As shown, the ECG curve may illustrate several cycles of the heart as the heart beats during the procedure. Each cycle may include a P wave representative of the depolarization of the atria, a QRS complex which represents the depolarization of the ventricles, and a T wave which represents the repolarization of the ventricles. As shown in FIG. 11, the curve 1110 illustrate a constant heart rate. For example, a region of the curve 1110 at one part of the cycle, may be repeated in subsequent cycles and may be spaced apart by equal periods of time along the curve. As an example, a point 1112 is shown on the ECG curve 1110. The point 1112 may be referred to as a feature of a cyclic waveform. This point 1112 may correspond to the peak of the R wave, e.g., a maximum voltage detected by the ECG system for a given heartbeat cycle. The point 1112 may be associated with the time at which a pulse wave leaves the heart. The point 1112 may be associated with any other time, e.g., any identifiable feature of the ECG signal (including without limitation, the start of a P-wave, the peak of a P-wave, the end of a P-wave, a PR interval, a PR segment, the beginning of a QRS complex, the start of an R-wave, the peak of an R-wave, the end of an R-wave, the end of a QRS complex (J-point), an ST segment, the start of a T-wave, the peak of a T-wave, and the end of a T-wave). In some examples, the point 1112 may correspond to a time value along the time axis 1140. The time value corresponding to the point 1112 may be the time TI_{A} 840 described with reference to FIG. 8.

The ECG curve 1110 may be associated with an axis 1180. The axis 1180 may illustrate a range of values associated with the ECG curve 1110. For example, the axis 1180 may correspond to units of voltage, including values in millivolts or other values or units.

Additionally shown in FIG. 11, is the is the pressure curve 1120. The pressure curve 1120 may be associated with the same time axis 1140. The pressure curve 1120 may be referred to as a cyclic waveform. A point 1114 along the pressure curve 1120 may reflect a minimum pressure value detected by a pressure sensor (e.g., the sensor 804 described with reference to FIGS. 8-10) for a given cycle. The point 1114 may be referred to as a feature of the cyclic waveform. The pressure curve 1120, like the ECG curve 1110, may include multiple cycles corresponding to the heartbeat, and/or more specifically, pulse waves sent from the heart of the patient anatomy. In this way, just like the point 1112 of the ECG curve 1110, the pressure curve 1120 may include multiple points of minimum pressure similar to the point 1114 in subsequent cycles of the pressure curve 1120.

As shown in FIG. 11, the point 1114 of the pressure curve 1120 may be associated with a time value along the time axis 1140. In some embodiments, the point 1114 may be the time at which a pulse wave is received or measured by the pressure sensor (e.g., the pressure sensor 804). The point 1114 may be associated with any other time, e.g., any other identifiable features of the pressure curve 1120, such as a minimum value, a maximum value, the dicrotic notch, a start or an end of an up-slope, a start or an end of a down-slope, the location of a maximum up-slope, the location of a maximum down-slope. In some examples, the point 1114 may correspond to the time value T2_{A} 842 described with reference to FIG. 8.

The pressure curve 1120 may be associated with an axis 1182. The axis 1182 may illustrate a range of values associated with the pressure curve 1120. For example, the axis 1182 may correspond to units of pressure, including values in mmHg or other values or units.

In some embodiments, the processor circuit of the system 100 may determine the time value associated with the point 1112 of the ECG waveform 1110 and the time value of the point 1114 of the pressure curve 1120. A difference between these time values may be determined to calculate the time T_{A1} showing FIG. 11. In some embodiments, the time T_{A1} may correspond to the time T_{A} described with reference to FIG. 8.

As shown in FIG. 11, the processor circuit may identify a point such as the point 1112 in any of the cycles of the ECG waveform 1110. Similarly, the processor circuit may identify a point similar to the point 1114 in any of the cycles of the pressure curve 1120. Based on any of these identified locations along the time axis 1140 a time similar to the time Tai may be calculated for any of the cycles of the ECG waveform 1110 or the pressure curve 1120. As an example, shown in FIG. 11, a point 1115 similar to the point 1112 may be identified and shown in the ECG curve 1110 and a point 1116 similar to the point 1114 may be shown and identified in the pressure curve 1120. A difference in the time values associated with these points yields an additional time value T_{A2}. The time value T_{A2} may be substantially similar to time value T_{A1}. However, in some embodiments, in particular if the heart rate of the patient is not constant but varies slightly, there may be a difference in the value of time T_{A2} and the time T_{A1}. Similarly, as shown in FIG. 11, a point 1117 and a point 1118 of the curves 1110 and 1120 respectively may be determined and an additional time T_{A3} may be calculated. Similarly, the time T_{A3} may vary from both the times T_{A1} and T_{A2} or may be the same or similar.

Any of the times T_{A1}, T_{A2}, and/or T_{A3} shown in FIG. 11 may correspond to the time it takes for a pulse wave to travel from the heart to the location of the pressure sensor within the renal artery of the patient. In some embodiments, the processor circuit may be configured to acquire multiple time measurements, such as the times T_{A1}, T_{A2}, and/or T_{A3}, to enhance the accuracy of the time value corresponding to the duration of time it takes for a pulse wave to travel from the heart to the location of the pressure sensor. In some embodiments, the times T_{A1}, T_{A2}, and/or T_{A3}, may be averaged and defined as the time it takes for the pulse wave to travel from the heart to the location of the pressure sensor. Any suitable number of times based on points of the curve 1110 in the pressure curve 1120, as shown in FIG. 11, may be determined based on the duration of time at which the pressure sensor remains stationary at one position within the renal artery. In some aspects, the time delay for the blood wave to travel from the heart to the sensor, or the time delay required for the blood wave to travel from two positions within the renal artery may be calculated in other ways. For example, the time delay may be determined from frequency domain measurements, such as via a fast fourier transform (FFT) and be determined based on the phase difference of the two signals at the heart frequency.

FIG. 12 is a diagrammatic view of an ECG curve 1210 and a blood pressure curve 1220 associated with a time axis 1240 and acquired after an intravascular device is moved within a renal artery, according to aspects of the present disclosure.

Similar to FIG. 11, FIG. 12 may illustrate two plots of data corresponding to the measurements and calculations described with reference to FIG. 9. The ECG data and intravascular pressure data shown in FIG. 12 may similarly be obtained simultaneously. In that regard, the processor circuit of the system 100 may be configured to synchronize intravascular data, including pressure, flow, etc., with ECG data. It is noted that the position of the pressure curve 1220 may be shifted in time in relation to the ECG curve 1210 compared to the position of the pressure curve 1120 in relation to the ECG curve 1110. This shift may be due to the position of the pressure sensor while acquiring the data of FIG. 12 being proximal to the position of the pressure sensor while acquiring the data of FIG. 11. For example, referring to FIG. 10, the position of the sensor for the data of FIG. 11 may be the location 891 and the position of the sensor for the data of FIG. 12 may be the location 991. In this way, the distance from the heart to the position 991 may be less than the distance from the heart to the position 891. As a result, the duration of time needed for a blood pulse to travel from the heart to the position 991 may be less than the time needed to travel from the heart to the position 891. This shortened time may result in the shift of the pressure curve 1220 shown in FIG. 12. This shift is also illustrated by the arrow 1290.

As an example, the ECG system described previously may acquire data such as the ECG curve 1210 shown in FIG. 12 The ECG curve 1210 may correspond to the movement of the heart 830 (FIGS. 8-10) during a pulse wave velocity measurement procedure. The ECG curve 1210 may be referred to as a cyclic waveform. As shown, the ECG curve 1210 may illustrate several cycles of the heart as the heart beats during the procedure. Each cycle may include any of the depolarization and/or repolarization cycles described with reference to FIG. 11. The curve 1210 illustrate a constant heart rate or a varying heart rate. Similar to the identification of points along the ECG curve 1110 (e.g., the points 1112, 1115, and 1117 of FIG. 11), the system may identify points along the ECG curve 1210. For example, a point 1212 may correspond to a time value of maximum voltage of a cardiac cycle. The point 1212 may be referred to as a feature of a cyclic waveform. The point 1212 may be associated with the time at which a pulse wave leaves the heart or any other time. In some examples, the point 1212 may correspond to a time value along the time axis 1240. The time value corresponding to the point 1212 may be the time T1B 940 described with reference to FIG. 9.

Additionally shown and FIG. 12, is the is the pressure curve 1220. The pressure curve 1220 may be associated with the same time axis 1240. The pressure curve 1220 may be referred to as a cyclic waveform. A point 1214 along the pressure curve 1220 may reflect a minimum pressure value detected by a pressure sensor (e.g., the sensor 804) for a given cardiac cycle. The point 1214 may be referred to as a feature of a cyclic waveform. The pressure curve 1220, like the ECG curve 1210, may include multiple cycles corresponding to the heartbeat, or more specifically, pulse waves sent from the heart of the patient anatomy. In this way, just like the point 1212 of the ECG curve 1210, the pressure curve 1220 may include multiple points of minimum pressure similar to the point 1214 in subsequent cycles of the pressure curve 1220.

As shown in FIG. 12, the point 1214 of the pressure curve 1220 may be associated with a time value along the time axis 1240. In some embodiments, the point 1214 may be the time at which a pulse wave is measured by the pressure sensor (e.g., the pressure sensor 804). the point 1214 may be associated with any other time. In some examples, the point 1214 may correspond to the time value T_{2B} 942 described with reference to FIG. 9.

In some embodiments, the processor circuit of the system 100 may determine the time value associated with the point 1212 of the ECG waveform 1210 and the time value of the point 1214 of the pressure curve 1220. A difference between these time values may be determined to calculate the time T_{B1} shown in FIG. 12. In some embodiments, the time T_{B1} may correspond to the time T_{B} described with reference to FIG. 9.

As shown in FIG. 12, the processor circuit may identify a point such is the point 1212 in any of the cycles of the ECG waveform 1210. Similarly, the processor circuit may identify a point similar to the point 1214 in any of the cycles of the pressure curve 1220. Based on any of these identified locations along the time axis 1240 a time similar to the time T_{B1} may be calculated for any of the cycles of the ECG waveform 1210 or the pressure curve 1220. As an example, shown in FIG. 12, a point 1215 similar to the point 1212 may be identified and shown in the ECG curve 1210 and a point 1216 similar to the point 1214 may be shown how identified in the pressure curve 1220. A difference in the time values associated with these points may yield an additional time value T_{A2}. The time value T_{A2} may be substantially similar to time value T_{A1}. However, in some embodiments, in particular if the heart rate of the patient is not constant but varies slightly, there may be a difference in the value of time T_{A2} and the time T_{A1}. Similarly, as shown in FIG. 12, a point 1217 at a point 1218 of the curves 1210 and 1220 respectively may be determined and an additional time T_{B3} may be calculated. Similarly, the time T_{B3} may vary from both the times TB1 and TB2 or may be the same or similar.

Any of the times T_{B1}, T_{B2}, and/or T_{B3} shown in FIG. 12 may correspond to the time it takes for a pulse wave to travel from the heart to the location of the pressure sensor within the renal artery of the patient. In some embodiments, the processor circuit may be configured to acquire multiple time measurements, such as the times T_{B1}, T_{B2}, and/or T_{B3}, to enhance the accuracy of the time value corresponding to the amount of time it takes for a pulse wave to travel from the heart to the location of the pressure sensor. In some embodiments, the times T_{B1}, T_{B2}, and/or T_{B3}, may be averaged and defined as the time it takes for the pulse wave to travel from the heart to the location of the pressure sensor. Any suitable number of times based on points of the curve 1210 in the pressure curve 1220, as shown in FIG. 12, may be determined based on the amount of time at which the pressure sensor remains stationary at one position within the renal artery.

As described with reference to FIG. 10 previously, the time T_{B} may be subtracted from the time T_{A} to yield a time T 1042 corresponding to the time for a pulse wave to travel from the location 991 to the location 891 within the renal artery (see FIG. 10). This time T 1042 and the distance 1040 between the locations 891 and 991 are used to determine the velocity of the pulse wave between the locations 891 and 991, and by extension, the pulse wave velocity in the renal artery. Similarly, with reference to FIG. 11 and FIG. 12, the times calculated may be used to determine pulse wave velocity. For example, the time T_{B1} (FIG. 12) may be subtracted from the time T_{A1} (FIG. 11) to calculate a time of a pulse wave to travel from the location at which the data of FIG. 12 was to the location at which the data of FIG. 11 was acquired. In other embodiments, an average of all times of FIG. 12 (e.g., the times T_{B1}, T_{B2}, T_{B3}, etc.) may be subtracted from an average of all the times of FIG. 11 (e.g., the times T_{A1}, T_{A2}, T_{A3}, etc.). This time value may also be used as the time for a pulse wave to travel from the location of the sensor of FIG. 12 to the location of the sensor of FIG. 11.

As previously mentioned, the device 802 shown in FIGS. 8-10 may be any suitable device. As a result, the data corresponding to the curves 1120 and 1220 of FIGS. 11 and 12 respectively may also be any suitable data. Specifically, although the sensor 804 of the device 802 was described as a pressure sensor with reference to FIGS. 8-10, it may alternatively be a blood flow sensor or an intravascular imaging assembly or sensor. Similarly, although the curves 1120 and 1220 of FIGS. 11 and 12 were described as pressure curves, they may alternatively be curves illustrating blood flow data over time or the diameter or cross-sectional area of the renal artery over time. It is noted that the ECG curve 1110 of FIG. 11 and the ECG curve 1210 of FIG. 12 may be portions of the same ECG curve. For example, one ECG curve may be based on ECG data received by the heart monitor throughout an entire procedure. The curve 1110 may correspond to a portion of that ECG curve through one period of time of the procedure and the curve 1210 may correspond to a portion of that ECG curve through another period of time of the procedure that is earlier or later than the time period of the curve 1110. Similarly, the pressure curve 1120 of FIG. 11 and the pressure curve 1220 of FIG. 12 may be portions of the same pressure curve. For example, one pressure curve may be based on pressure data received by the intraluminal device throughout an entire procedure. The curve 1120 may correspond to a portion of that pressure curve through one period of time of the procedure and the curve 1220 may correspond to a portion of that pressure curve through another period of time of the procedure that is earlier or later than the time period of the curve 1120.

The system 100 (e.g., a processor circuit of the system 100) may be configured to output to a display (e.g., the display 160) any data or results acquired or generated by the system 100. For example, a numerical value of time T 1042, a distance (e.g., a distance between the locations 891 and 991), a pulse wave velocity or any other numerical value. Alternatively, any of these values may be displayed graphically, such as an indicator on a plot, chart, or in any other way. In some aspects, the processor circuit of the system 100 may output to the display acquired ECG curves or intravascular curves (e.g. FIG. 11 or 12).

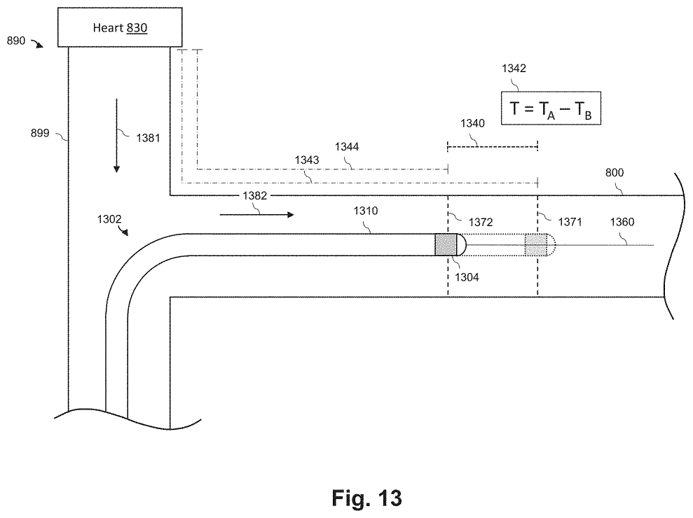

FIG. 13 is a diagrammatic view of an intravascular device within a region of a patient anatomy showing the heart 830, aorta 899, and renal artery 800, according to aspects of the present disclosure.

At a step of the present disclosure, an intravascular device 1302 may be positioned within the patient vasculature. The intravascular device 1302 may be an intravascular imaging device. In some embodiments, the intravascular device 1302 may be an optical coherence tomography (OCT) device including an OCT imaging assembly with an optical lens, optical fiber, or any other suitable components, an intravascular photoacoustic (IVPA) device, an intravascular ultrasound (IVUS) device including a single transducer or a transducer array, or any other type of imaging device. In some embodiments, the device 1302 may be a solid-state IVUS imaging device including an IVUS imaging assembly including multiple ultrasound transducers configured to emit and receive ultrasound energy positioned circumferentially around the assembly. In some embodiments, the device 1302 may be a rotational IVUS imaging device including a single ultrasound transducer configured to emit and receive ultrasound energy and to be rotated circumferentially around the assembly.

In some embodiments, the IVUS imaging device 1302 may include a guidewire 1460. In some embodiments, the guidewire 1460 may be positioned at an initial step of the present disclosure within the renal artery 800. The IVUS imaging device may additionally include a flexible elongate member 1410 and an IVUS transducer array 1304. The flexible elongate member 1410 and IVUS imaging assembly 1304 be referred to as an IVUS imaging catheter. The IVUS imaging catheter may define a central lumen through which the guidewire 1460 may be received. The IVUS catheter may thus be positioned around the guidewire 1460 and guided to a desired location within the renal artery 800.

As shown in FIG. 13, the device 1302 may be positioned such that ultrasound transducer array 1304 of the device is positioned within a renal artery 800 of the patient vasculature. The ultrasound transducer array 1304 of the device 1302 may be positioned at the location 1371 within the renal artery 800. After the ultrasound transducer array 1304 is positioned at the location 1371, the user of the system, or a processor circuit of the system (e.g., the processor circuit 410), may direct the array 1304 to begin acquiring intravascular imaging data. This imaging data may be used to generate IVUS images of the vessel 800. As the array 1304 receives or acquires the intravascular imaging data, a blood pulse wave may travel in a downward direction as shown by the arrow 1381 and then into the renal artery as shown by the arrow 1382. As this pulse wave passes the ultrasound transducer array 1304, intravascular images acquired by the IVUS device may show a change in the diameter of the renal artery 800.

Similar to the timing of pulse waves as monitored by the ECG system and the pressure device as described with reference to FIGS. 8-10, one or more blood pulse waves may be sent from the heart 830 to the rest of the body. With the transducer array 1304 held stationary at the location 1371, the time at which a pulse leaves the heart 830 may be recorded. The time at which the wave is observed by the IVUS imaging device 1302 may also be recorded. These two times may determine a time T_{A}.

At a subsequent step, the IVUS imaging array may be moved to a location 1372 and the process may be repeated. Specifically, a time at which a pulse leaves the heart 830 may be record and the time at which it is observed by the IVSU imaging device 1302 may be recorded. These times may determine an amount of time T_{B}.

The time T_{A} may correspond to the distance shown by the indicator 1343. This distance may be the distance traveled by a blood pulse wave from the heart 830 to the location 1371. Similarly, the time T_{B} may correspond to the distance shown by the indicator 1344. This distance may be the distance traveled by a blood pulse wave from the heart 830 to the location 1372.

The times T_{A} and T_{B} may be compared (e.g., a difference between the two times T_{A} and T_{B} may be calculated) to calculate a time T 1342 shown. The time T 1342 may correspond to the amount of time it takes for a blood pulse wave to travel from the position 1372 to the position 1371. In addition, based on coregistration of the locations of the IVUS imaging device with an extraluminal image, the positions of 1371 and 1372 may be known and a distance between these positions (e.g., the distance 1340) may be calculated. Based on the distance 1340 and the time T 1342, a velocity of a blood pulse wave within the renal artery may be calculated. Specifically, the pulse wave velocity through the renal artery 800 as shown in FIG. 13 may be determined by dividing the distance 1340 by the time T 1342.

It is noted, that plots similar to those shown and described with reference to FIGS. 11 and 12 may also be generated corresponding to the embodiment described in FIG. 13. For example, a plot with an ECG curve (e.g., similar to the ECG curves 1110 and 1210 of FIGS. 11 and 12) and a renal artery diameter curve may be calculated and displayed in association with a time axis. In general, any suitable intravascular length or distance can be used (e.g., average diameter, minimum diameter, maximum diameter, etc.). In some embodiments, the renal artery diameter curve may be similar to the blood pressure curve 1210 shown and described with reference to FIG. 12 in that it may represent a cyclical waveform (also referred to as a periodic wave form) corresponding to the cardiac cycle. However, the renal artery diameter may reflect the observed diameter of the vessel wall, or a lumen of the renal artery over time. As an example, the renal artery may expand as a blood pulse passes through the renal artery. In some embodiments, just as the processor circuit 410 (FIG. 4) may select a point along the pressure curve 1120 (FIG. 11) corresponding to a minimum pressure, the processor circuit 410 (FIG. 4) may select a point along the renal artery diameter curve corresponding to a maximum, minimum, or any other identifiable point. As described with reference to FIG. 11, a selected point of the renal artery diameter curve may be compared to a point of the ECG both for data received at the position 1371 and the position 1372. These times may be compared (e.g., subtracted) to yield an amount of time it takes for a blood pulse wave to travel from the position 1372 to the position 1371 of the renal artery.

In the case in which a flow sensor is placed at the distal most portion of the intravascular device, similar plots as those described with reference to FIG. 11 and FIG. 12 may also be generated and displayed. In some embodiments, the shape of the plot corresponding to flow data may differ from the shape of a pressure plot. However, the plot associated with flow data may also exhibit cyclical wave-like patterns corresponding to the cardiac cycle. Because of this cyclical nature, points on the flow data plot may be selected and compared with points on the ECG curve. As described with reference to FIGS. 11 and 12, these points may be compared to determine the time it takes for a pulse wave to move from one location within the renal artery to another (e.g., from the position 1372 to the position 1371).

In some embodiments, the flow sensor of an intravascular device including a flow sensor may be substantially similar to the flow sensor described with reference to FIG. 3.

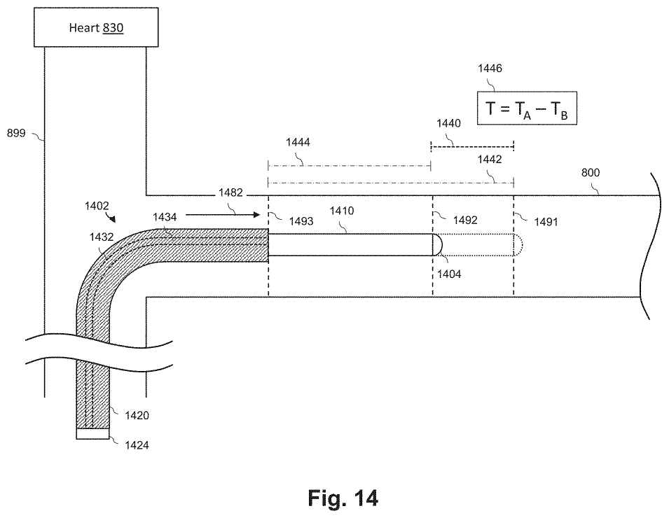

FIG. 14 is a diagrammatic view of an intravascular device within a region of a patient anatomy showing the heart 830, aorta 899, and renal artery 800, according to aspects of the present disclosure.

FIG. 14 may illustrate an embodiment of the present disclosure in which a device 1402 including two pressure sensors may be used to determine the pulse wave velocity of a blood pulse within the renal artery.

As shown in FIG. 14, the device 1402 may include a pressure sensor 1404 and a pressure sensor 1424. In some embodiments, the sensor 1404 may be a different type of sensor, including a flow sensor and/or an intravascular imaging sensor like those described herein.

In some embodiments, the distal sensor 1404 may acquire data within the renal artery 800. For example, the distal sensor 1404 may be moved to any position within the renal artery 800 including the position 1491 and/or the position 1492.

In some embodiments, the pressure sensor 1424 may be configured to measure the pressure of blood at the location 1493. In the embodiment shown in FIG. 14, the pressure sensor 1424 may be positioned at a location proximal to the distal sensor 1404. For example, in some embodiments, the pressure sensor 1424 may be positioned outside the patient body during a nerve stimulation or nerve ablation procedure. The pressure sensor 1424 may be at a proximal end of a flexible elongate member 1420. As shown in FIG. 14, the flexible elongate member 1420 may be inserted within the vessel of the patient. The flexible elongate member 1420 may be configured to define one or more inner lumens. As an example, the flexible elongate member 1420 may define an inner lumen 1432. In some embodiments, the proximal end of the lumen 1432 may terminate at the pressure sensor 1424. In this way, the pressure sensor 1424 may be configured to monitor pressure measurements of a fluid 1434 within the lumen 1432. In some embodiments, blood from the renal artery 800 may enter the lumen 1432 at a distal end of the lumen 1432. In this way, blood from the patient may fill the lumen 1432 extending to the proximal end by the pressure sensor 1424. In some aspects, the lumen 1432 may be filled with a saline or any other fluid that is completely or nearly incompressible. The pressure sensor 1424 may then monitor the pressure of blood within the renal artery 800. In some embodiments, the lumen 1432 may be a closed chamber. For example, the device 1402 may include a barrier at the distal end of the lumen 1432 separating blood from the vessel 800 from a fluid 1434 within the lumen 1432. In such an embodiment, the barrier at the distal end of the lumen 1432 may be any suitable barrier. The barrier may allow pressure from the blood of the vessel 800 to compress the fluid 1434 within the lumen 1432. In this way, the pressure of the fluid 1434 within the lumen 1432 may be the same as the pressure of the blood within the vessel 800. The pressure sensor 1424 may then measure the pressure of the fluid 1434 within the lumen 1432. This pressure may be conveyed to the system as the blood pressure of the renal artery 800 measured at the location 1493. In some aspects, the tip catheter may also be positioned in another vessel (other the renal artery), such as the aorta (e.g., abdominal aorta).

At a step of the present disclosure, the intravascular device 1402 may be positioned within the patient vasculature. The intravascular device 1402 may include the proximal pressure sensor 1424 and a distal pressure sensor 1404. Proximal pressure sensor 1424 can also be referenced as an aortic pressure sensor. In some embodiments, the sensor 1404 may be any type of sensor, including a flow sensor, a pressure sensor, or an imaging sensor, such as an optical coherence tomography (OCT) device, an intravascular photoacoustic (IVPA) device, an intravascular ultrasound (IVUS) device, or any other type of imaging device. For example, as described with reference to FIGS. 11 and 12, a pressure curve may be displayed corresponding to the proximal pressure sensor 1424. An additional curve may be displayed corresponding to the distal sensor 1404. In some embodiments, this additional curve may be a pressure curve (e.g., if the sensor 1404 is a pressure sensor), a flow curve showing the velocity of blood over time (e.g., if the sensor 1404 is a blood velocity/flow sensor), or a diameter curve showing the diameter of the vessel as observed by the sensor (e.g., if the sensor 1404 is an intravascular imaging device). Just as points between the ECG curve 1110 and the pressure curve 1120 may be compared to calculate various time values (see FIG. 11), points between the pressure curve of the proximal sensor 1424 and the curve of intraluminal data (e.g., pressure, flow, or diameter) may be compared to also determine similar time values. Subsequently, as previously described, these time values may be compared with other time values with the distal sensor at a new location and a pulse wave velocity may be calculated by dividing the distance between the two locations with the time difference calculated.

In some embodiments, the device 1402 may include a guidewire around which the sensor 1404, flexible elongate member 1410, and/or flexible elongate member 1420 may be positioned. In some embodiments, the flexible elongate member 1420 with its defined lumen 1432 and pressure sensor 1424 may be referred to as a guide catheter.

As shown in FIG. 14, the device 1402 may be positioned such that the sensor 1404 of the device is positioned within a renal artery 800 of the patient vasculature. The sensor 1404 of the device 1402 may be positioned at the location 1491 within the renal artery 800. After the sensor 1404 is positioned at the location 1491, the user of the system, or a processor circuit of the system (e.g., the processor circuit 410), may direct the sensor 1404 and the sensor 1424 to begin acquiring intravascular data. As the sensor 1404 and the sensor 1424 acquire the intravascular data, a blood pulse wave may travel in a downward direction and then along the renal artery as shown by the arrow 1482. As this pulse wave passes the sensor the position 1493, the pressure sensor 1424 may observe a change in the pressure. Similarly, as the pulse passes the sensor 1404, it may be measured by the sensor 1404 (e.g., as a change in pressure for a pressure sensor, a change in flow for a flow sensor, or a change in vessel or lumen diameter for an imaging sensor).

With the transducer array held stationary at the location 1491, the time at which a pulse is detected at the position 1493 by the sensor 1424 may be recorded. Similarly, the time at which the wave is observed by the distal sensor 1404 may also be recorded. These two times may determine a time T_{A}.

At a subsequent step, the distal sensor 1404 may be moved to a location 1492 and the process may be repeated. Specifically, a time at which a pulse is measured by the sensor 1424 may be recorded and the time at which it is observed by the distal sensor 1424 may be recorded. These times may determine an amount of time T_{B}.

The time T_{A} may correspond to the distance shown by the indicator 1442. This distance may be the distance traveled by a blood pulse wave from the location 1493 to the location 1491. Similarly, the time T_{B} may correspond to the distance shown by the indicator 1444. This distance may be the distance traveled by a blood pulse wave from the location 1493 to the location 1492.

The times T_{A} and T_{B} may be compared (e.g., a difference between the two times T_{A} and T_{B} may be calculated) to calculate a time T 1446 shown. The time T 1446 may correspond to the amount of time it takes for a blood pulse wave to travel from the position 1492 to the position 1491. In addition, based on coregistration of the locations of the distal sensor 1404 with an extraluminal image, the positions of 1491 and 1492 may be known and a distance between these positions (e.g., the distance 1440) may be calculated. Based on the distance 1440 and the time T 1446, a velocity of a blood pulse wave within the renal artery may be calculated. Specifically, the pulse wave velocity through the renal artery 800 as shown in FIG. 14 may be determined by dividing the distance 1440 by the time T 1446.

It is noted, that plots similar to those shown and described with reference to FIGS. 11 and 12 may also be generated corresponding to the embodiment described in FIG. 14. However, plots associated with the measurements described in FIG. 14 may not include an ECG curve because the ECG curve may not be measured. In one example, rather than an ECG curve (e.g., similar to the ECG curves 1110 and 1210 of FIGS. 11 and 12) a pressure curve corresponding to pressure data acquired by the sensor 1424 may be calculated and displayed. A pressure curve, flow curve, or vessel diameter curve (depending on the type of sensor used for the distal sensor 1404) may also be calculated and displayed in association with a time axis. In some embodiments, the data curve of the distal sensor 1404 may be similar to the blood pressure curve 1210 shown and described with reference to FIG. 12 in that it may represent a cyclical waveform corresponding to the cardiac cycle, or may be similar to any of the other data curves described herein. The processor circuit 410 (FIG. 4) may be configured to identify a point of each cardiac cycle within the pressure curve acquired by the sensor 1424. This may be a maximum pressure, a minimum pressure, or some other point. The processor circuit may find a similar point of the curve acquired by the distal sensor 1404. This point may be a maximum value, a minimum value, or any other value. The time between these selected points may be determined for the data acquired at the position 1491 and may be shown in FIG. 14 as time T_{A}. The same procedure may be followed for the data acquired at the position 1492 and may be shown in FIG. 14 as time T_{B}. These two times may be subtracted from one another to yield the time T 1446. Based on the calculation of the value time T 1446 and the distance value 1440, the velocity of a pulse within the renal artery 800 may be determined.

In some embodiments, the time T_{A} corresponding to the time for a pulse wave to travel from the location 1393, as measured by the pressure sensor 1424, to the location 1491, as measured by the distal sensor 1404, may be compared with a distance measurement between the location 1493 and 1491 (e.g., the distance 1442). This distance measurement 1442 may be determined based on coregistration. In some embodiments, the distance 1442 may be divided by the time T_{A} to yield a velocity of a pulse wave within the renal artery 800. Similarly, the distance 144 and the time T_{B} may be used to determine the pulse wave velocity.

In some aspects, additional metrics or calculations may be displayed to a user. For example, a pulse wave velocity map may be created. A pulse wave velocity map may include a view of one or more vessels in an extraluminal image, which includes overlaid pulse wave velocity measurements at different locations along the one or more vessels. The extraluminal image can be or be based on an x-ray image (angiography image, angiography image on a registered 3D image from rotational angio or CT/MR), CT image, MR image, etc. For example, pulse wave velocity measurements at the different locations may be displayed as values, symbols, colors, or any suitable graphical representation overlaid on the image. In some aspects, these graphical representations related to pulse wave velocity measurements may overlay the image between the locations of measurement (e.g., locations 1371 and 1372 of FIG. 13 or locations 1491 and 1492 of FIG. 14).

The velocity of the pressure/flow pulse (pulse wave velocity or PWV) inside the main renal artery may be indicative of the outcome of renal denervation. As a result, determining pulse wave velocity may be useful for patient stratification for renal denervation. Pulse wave velocity can be predictive of the effective of renal denervation on a patient. Thus, determining pulse wave velocity accurately and quickly, leveraging co-registration, advantageously improves a physician's ability to determine treatment for the patient, leading to better physiological outcomes for the patient.

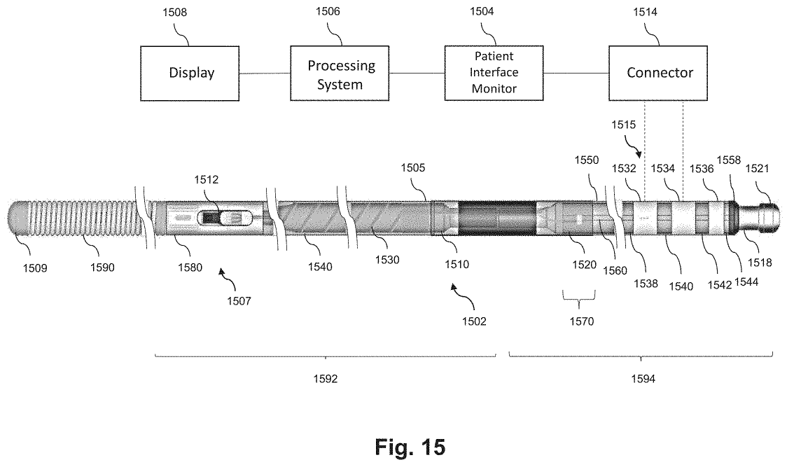

FIG. 15 is a diagrammatic side view of an intraluminal (e.g., intravascular) sensing system 100 that includes an intravascular device 1502 comprising conductive members 1530 (e.g., a multi-filar electrical conductor bundle) and conductive ribbons 1560, according to aspects of the present disclosure. The intravascular device 1502 can be an intravascular guidewire sized and shaped for positioning within a vessel of a patient. The intravascular device 1502 includes a distal tip 1509 (e.g., an atraumatic distal tip) and an electronic component 1512. For example, the electronic component 1512 can be a pressure sensor and/or flow sensor configured to measure a pressure of blood flow within the vessel of the patient, or another type of sensor including but not limited to a temperature or imaging sensor, or combination sensor measuring more than one property. For example, the pressure data obtained by a pressure sensor can be used to calculate physiological variables such as a pressure ratio (e.g., fractional flow reserve (FFR), instantaneous wave free ratio (iFR), Pd/Pa, etc.). For example, the flow data obtained by a flow sensor can be used to calculate physiological variables such as coronary flow reserve (CFR). The intravascular device 1502 includes a flexible elongate member 1505. The electronic component 1512 is disposed at a distal portion 1507 of the flexible elongate member 1505. The electronic component 1512 can be mounted at the distal portion 1507 within a housing 1580 in some embodiments. A flexible tip coil 1590 extends distally from the housing 1580 at the distal portion 1507 of the flexible elongate member 1505. A connection portion 1515 located at a proximal end of the flexible elongate member 1505 includes conductive portions 1532, 1534. In some embodiments, the conductive portions 1532, 1534 can be conductive ink that is printed and/or deposited around the connection portion 1515 of the flexible elongate member 1505. In some embodiments, the conductive portions 1532, 1534 are conductive, metallic bands or rings that are positioned around the flexible elongate member. A locking area is formed by a collar or locking section 1518 and knob or retention section 1521 are disposed at the proximal portion 109 of the flexible elongate member 1505.

The intravascular device 1502 in FIG. 15 includes core wire comprising a distal core 1510 and a proximal core 1520. The distal core 1510 and the proximal core 1520 are metallic components forming part of the body of the intravascular device 1502. For example, the distal core 1510 and the proximal core 1520 may be flexible metallic rods that provide structure for the flexible elongate member 1505. The distal core 1510 and/or the proximal core 1520 can be made of a metal or metal alloy. For example, the distal core 1510 and/or the proximal core 1520 can be made of stainless steel, Nitinol, nickel-cobalt-chromium-molybdenum alloy (e.g., MP35N), and/or other suitable materials. In some embodiments, the distal core 1510 and the proximal core 1520 are made of the same material. In other embodiments, the distal core 1510 and the proximal core 1520 are made of different materials. The diameter of the distal core 1510 and the proximal core 1520 can vary along their respective lengths. A joint between the distal core 1510 and proximal core 1520 is surrounded and contained by a hypotube. The electronic component 1512 can in some cases be positioned at a distal end of the distal core 1510.

In some embodiments, the intravascular device 1502 comprises a distal subassembly and a proximal subassembly that are electrically and mechanically joined together, which creates an electrical communication between the electronic component 1512 and the conductive portions 1532, 1534. For example, flow data obtained by the electronic component 1512 (in this example, electronic component 1512 is a flow sensor) can be transmitted to the conductive portions 1532, 1534. In an exemplary embodiment, the flow sensor 1512 is a single ultrasound transducer element. In some embodiments, the transducer element emits ultrasound signals, receives echoes, and generates electrical signals representative of the echoes. The processing system 1506 processes the electrical signals to extract the flow velocity of the fluid. In some embodiments, the electronic component is a pressure transducer (e.g., based on piezoresistive technology) and generates electrical signals representative of the pressure within the vessel. The signal carrying filars carry these electrical signals from the sensor at the distal portion to the connector at the proximal portion.

Control signals from a processing system 1506 (e.g., a processor circuit of the processing system 1506) in communication with the intravascular device 1502 can be transmitted to the electronic component 1512 via a connector 1514 that attached to the conductive portions 1532, 1534. The distal subassembly can include the distal core 1510. The distal subassembly can also include the electronic component 1512, the conductive members 1530, and/or one or more layers of insulative polymer/plastic 1540 surrounding the conductive members 1530 and the core 1510. For example, the polymer/plastic layer(s) can insulate and protect the conductive members of the multi-filar cable or conductor bundle 1530. The proximal subassembly can include the proximal core 1520. The proximal subassembly can also include one or more polymer layers 1550 (hereinafter polymer layer 1550) surrounding the proximal core 1520 and/or conductive ribbons 1560 embedded within the one or more insulative and/or protective polymer layer 1550. In some embodiments, the proximal subassembly and the distal subassembly are separately manufactured. During the assembly process for the intravascular device 1502, the proximal subassembly and the distal subassembly can be electrically and mechanically joined together. As used herein, flexible elongate member can refer to one or more components along the entire length of the intravascular device 1502, one or more components of the proximal subassembly (e.g., including the proximal core 1520, etc.), and/or one or more components the distal subassembly 1592 (e.g., including the distal core 1510, etc.). Accordingly, flexible elongate member may refer to the combined proximal and distal subassemblies described above. The joint between the proximal core 1520 and distal core 1510 is surrounded by the hypotube 215.

In various embodiments, the intravascular device 1502 can include one, two, three, or more core wires extending along its length. For example, a single core wire can extend substantially along the entire length of the flexible elongate member 1505. In such embodiments, a locking section 1518 and a section 1521 can be integrally formed at the proximal portion of the single core wire. The electronic component 1512 can be secured at the distal portion of the single core wire. In other embodiments, such as the embodiment illustrated in FIG. 15, the locking section 1518 and the section 1521 can be integrally formed at the proximal portion of the proximal core 1520. The electronic component 1512 can be secured at the distal portion of the distal core 1510. The intravascular device 1502 includes one or more conductive members 1530 (e.g., a multi-filar conductor bundle or cable) in communication with the electronic component 1512. For example, the conductive members 1530 can be one or more electrical wires that are directly in communication with the electronic component 1512. In some instances, the conductive members 1530 are electrically and mechanically coupled to the electronic component 1512 by, e.g., soldering. In some instances, the conductor bundle 1530 comprises two or three electrical wires (e.g., a bifilar cable or a trifilar cable). An individual electrical wire can include a bare metallic conductor surrounded by one or more insulating layers. The conductive members 1530 can extend along the length of the distal core 1510. For example, at least a portion of the conductive members 1530 can be spirally wrapped around the distal core 1510, minimizing or eliminating whipping of the distal core within tortuous anatomy.

The intravascular device 1502 includes one or more conductive ribbons 1560 at the proximal portion of the flexible elongate member 1505. The conductive ribbons 1560 are embedded within polymer layer 1550. The conductive ribbons 1560 are directly in communication with the conductive portions 1532 and/or 1534. In some instances, a multi-filar conductor bundle 1530 is electrically and mechanically coupled to the electronic component 1512 by, e.g., soldering. In some instances, the conductive portions 1532 and/or 1534 comprise conductive ink (e.g., metallic nano-ink, such as copper, silver, gold, or aluminum nano-ink) that is deposited or printed directed over the conductive ribbons 1560.

As described herein, electrical communication between the conductive members 1530 and the conductive ribbons 1560 can be established at the connection portion 1515 of the flexible elongate member 1505. By establishing electrical communication between the conductor bundle 1530 and the conductive ribbons 1560, the conductive portions 1532, 1534 can be in electrical communication with the electronic component 1512.

In some embodiments represented by FIG. 15, the intravascular device 1502 includes a locking section 1518 and a retention section 1521. To form locking section 1518, a machining process is used to remove polymer layer 1550 and conductive ribbons 1560 in locking section 1518 and to shape proximal core 1520 in locking section 1518 to the desired shape. As shown in FIG. 15, locking section 1518 includes a reduced diameter while retention section 1521 has a diameter substantially similar to that of proximal core 1520 in the connection portion 1515. In some instances, because the machining process removes conductive ribbons in locking section 1518, proximal ends of the conductive ribbons 1560 would be exposed to moisture and/or liquids, such as blood, saline solutions, disinfectants, and/or enzyme cleaner solutions, an insulation layer 1558 is formed over the proximal end portion of the connection portion 1515 to insulate the exposed conductive ribbons 1560.

In some embodiments, a connector 1514 provides electrical connectivity between the conductive portions 1532, 1534 and a patient interface monitor 1504. The Patient Interface Monitor 1504 may in some cases connect to a console or processing system 1506, which includes or is in communication with a display 1508.

The system 100 may be deployed in a catheterization laboratory having a control room. The processing system 1506 may be located in the control room. Optionally, the processing system 1506 may be located elsewhere, such as in the catheterization laboratory itself. The catheterization laboratory may include a sterile field while its associated control room may or may not be sterile depending on the procedure to be performed and/or on the health care facility. In some embodiments, device 1502 may be controlled from a remote location such as the control room, such that an operator is not required to be in close proximity to the patient.

The intraluminal device 1502, PIM 1504, and display 1508 may be communicatively coupled directly or indirectly to the processing system 1506. These elements may be communicatively coupled to the medical processing system 1506 via a wired connection such as a standard copper multi-filar conductor bundle 1530. The processing system 1506 may be communicatively coupled to one or more data networks, e.g., a TCP/IP-based local area network (LAN). In other embodiments, different protocols may be utilized such as Synchronous Optical Networking (SONET). In some cases, the processing system 1506 may be communicatively coupled to a wide area network (WAN).

The PIM 1504 transfers the received signals to the processing system 1506 where the information is processed and displayed (e.g., as physiology data in graphical, symbolic, or alphanumeric form) on the display 1508. The console or processing system 1506 can include a processor and a memory. The processing system 1506 may be operable to facilitate the features of the intravascular sensing system 100 described herein. For example, the processor can execute computer readable instructions stored on the non-transitory tangible computer readable medium.

The PIM 1504 facilitates communication of signals between the processing system 1506 and the intraluminal device 1502. The PIM 1504 can be communicatively positioned between the processing system 1506 and the intraluminal device 1502. In some embodiments, the PIM 1504 performs preliminary processing of data prior to relaying the data to the processing system 1506. In examples of such embodiments, the PIM 1504 performs amplification, filtering, and/or aggregating of the data. In an embodiment, the PIM 1504 also supplies high- and low-voltage DC power to support operation of the intraluminal device 1502 via the conductive members 1530.

A multi-filar cable or transmission line bundle 1530 can include a plurality of conductors, including one, two, three, four, five, six, seven, or more conductors. In the example shown in FIG. 15, the multi-filar conductor bundle 1530 includes two straight portions 232 and 236, where the multi-filar conductor bundle 1530 lies parallel to a longitudinal axis of the flexible elongate member 1505, and a spiral portion 234, where the multi-filar conductor bundle 1530 is wrapped around the exterior of the flexible elongate member 1505 and then overcoated with an insulative and/or protective polymer 1540. Communication, if any, along the multi-filar conductor bundle 1530 may be through numerous methods or protocols, including serial, parallel, and otherwise, wherein one or more filars of the bundle 1530 carry signals. One or more filars of the multi-filar conductor bundle 1530 may also carry direct current (DC) power, alternating current (AC) power, or serve as a ground connection.

The display 1508 may be a display device such as a computer monitor or other type of screen. The display 1508 may be used to display selectable prompts, instructions, and visualizations of imaging data to a user. In some embodiments, the display 1508 may be used to provide a procedure-specific workflow to a user to complete an intraluminal imaging procedure.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A system, comprising:
a processor circuit configured for communication with a display, a heart monitor, and an intravascular catheter or guidewire, wherein the processor circuit is configured to:
receive, from the intravascular catheter or guidewire, a first set of intravascular data obtained by a single intravascular sensor of the intravascular catheter or guidewire while the intravascular catheter or guidewire is positioned at a first location within the blood vessel;
receive, from the heart monitor, a first set of cardiovascular data obtained while the single intravascular sensor obtains the first set of intravascular data;
receive, from the intravascular catheter or guidewire, a second set of the intravascular data obtained by the single intravascular sensor while the intravascular catheter or guidewire is positioned at a second location within the blood vessel;
receive, from the heart monitor, a second set of the cardiovascular data obtained while the single intravascular sensor obtains the second set of intravascular data;
determine a distance between the first location and the second location;
determine a velocity of a pulse wave associated with blood flow within the blood vessel, based on the first set of intravascular data, the second set of intravascular data, the first set of cardiovascular data, the second set of cardiovascular data, and the distance; and
provide, to the display, an output based on the velocity of the pulse wave.

2. The system of claim 1, wherein the first set of the cardiovascular data and the second set of the cardiovascular data include electrocardiogram (ECG) data.

3. The system of claim 1,
wherein the single intravascular sensor comprises a pressure sensor, and
wherein the first set of the intravascular data and the second set of the intravascular data comprise intravascular pressure data.

4. The system of claim 1,
wherein the single intravascular sensor comprises a flow sensor, and
wherein the first set of the intravascular data and the second set of the intravascular data comprise intravascular flow data.

5. The system of claim 1,
wherein the single intravascular sensor comprises an imaging sensor, and
wherein the first set of the intravascular data and the second set of the intravascular data comprise intravascular imaging data.

6. The system of claim 1,
wherein the processor circuit is configured for communication with an extraluminal imaging device,
wherein the processor circuit is configured to receive one or more extraluminal images obtained by the extraluminal imaging device, and
wherein the processor circuit is configured to determine the distance based on the one or more extraluminal images.

7. The system of claim 1,
wherein the processor circuit is configured for communication with an extraluminal imaging device, and
wherein the processor circuit is configured to determine the distance based on co-registration of at least one of the first set of intravascular data or the second set of intravascular data to one or more extraluminal images obtained by the extraluminal imaging device.

8. The system of claim 1, wherein:
the first set of the cardiovascular data corresponds to a first cyclic waveform;
the first set of the intravascular data corresponds to a second cyclic waveform;
the second set of the cardiovascular data corresponds to a third cyclic waveform; and
the second set of the intravascular data corresponds to a fourth cyclic waveform.

9. The system of claim 8,
wherein the processor circuit is further configured to:
identify a first time at which a first feature of the first cyclic waveform occurs;
identify a second time at which a second feature of the second cyclic waveform occurs;
identify a third time at which a third feature of the third cyclic waveform occurs;
identify a fourth time at which a fourth feature of the fourth cyclic waveform occurs;
determine a first difference between the first time and the second time; and
determine a second difference between the third time and the fourth time, and
wherein the processor circuit is configured to determine the velocity of the pulse wave based on the first difference, the second difference, and the distance.

10. The system of claim 9,
wherein the processor circuit is configured to determine a third difference between the first difference and the second difference, and
wherein the processor circuit is configured to determine the velocity of the pulse wave based on the third difference and the distance.

11. The system of claim 10, wherein, to determine the velocity of the pulse wave, the processor circuit is configured to divide the distance by the third difference.

12. The system of claim 9,
wherein the first feature and the third feature comprise a same feature of the cardiovascular data, and
wherein the second feature and the fourth feature comprise a same feature of the intravascular data.

13. The system of claim 1, wherein the blood vessel comprises a renal artery.

14. A method, comprising:
receiving, by a processor circuit in communication with an intravascular catheter or guidewire comprising only a single intravascular sensor, a first set of intravascular data obtained by the single intravascular sensor while the intravascular catheter or guidewire is positioned at a first location within the blood vessel;
receiving, by the processor circuit, a first set of cardiovascular data while the single intravascular sensor obtains the first set of intravascular data, wherein the first set of cardiovascular data is obtained by a heart monitor in communication with the processor circuit;
receiving, by a processor circuit, a second set of the intravascular data obtained by the single intravascular sensor while the intravascular catheter or guidewire is positioned at a second location within the blood vessel;
receiving, by the processor circuit, a second set of the cardiovascular data obtained by the heart monitor while the single intravascular sensor obtains the second set of intravascular data;
determining, by the processor circuit, a distance between the first location and the second location;
determining, by the processor circuit, a velocity of a pulse wave associated with blood flow within the blood vessel, based on the first set of intravascular data, the second set of intravascular data, the first set of cardiovascular data, the second set of cardiovascular data, and the distance; and
providing, by the processor circuit, an output based on the velocity of the pulse wave to a display in communication with the processor circuit.

15. A system, comprising:
an intravascular catheter or guidewire configured to be positioned within a blood vessel of a patient and comprising only a single intravascular sensor; and
a processor circuit configured for communication with a heart monitor, an extraluminal imaging device, a display, and the intravascular catheter or guidewire, wherein the processor circuit is configured to:
determine a first time difference between when a first feature occurs in a first set of electrocardiogram (ECG) data and when a second feature occurs in a first set of intravascular data, wherein the first set of the intravascular data is obtained by the single intravascular sensor at a first location within the blood vessel simultaneously as the first set of the ECG data is obtained by the heart monitor;
determine a second time difference between when a third feature occurs in a second set of ECG data and when a fourth feature occurs in a second set of intravascular data, wherein the second set of the intravascular data is obtained by the single intravascular sensor at a second location within the blood vessel simultaneously as the second set of ECG data is obtained by the heart monitor;
determine a distance between the first location and the second location based on one or more extraluminal images obtained by the extraluminal imaging device;
determine a velocity of a pulse wave associated with blood flow within the blood vessel based on the distance, the first time difference, and the second time difference; and
provide, to the display, an output based on the velocity of the pulse wave.
